(19) United States
(12) Patent Application Publication
(10) Pub. No.: US 2025/0313890 A1
(43) Pub. Date: Oct. 9, 2025

(54) ANALYSIS OF ANALYTES AND SPATIAL GENE EXPRESSION

(71) Applicant: 10x Genomics, Inc., Pleasanton, CA (US)

(72) Inventors: Joakim Lundeberg, Stockholm (SE); Marco Vicari, Stockholm (SE); Mohammadreza Mirzazadeh, Stockholm (SE)

(21) Appl. No.: 19/242,457

(22) Filed: Jun. 18, 2025

Related U.S. Application Data
(63) Continuation of application No. PCT/US2023/085163, filed on Dec. 20, 2023.
(60) Provisional application No. 63/476,532, filed on Dec. 21, 2022.

(51) Int. Cl. C12Q 1/6872; C12Q 1/6806; G01N 1/30; G01N 1/34

(52) U.S. Cl. CPC C12Q 1/6872; C12Q 1/6806; G01N 1/30; G01N 1/34; G01N 2001/305

(57) ABSTRACT

Provided herein are methods for analyzing a biological sample that include contacting the biological sample with a non-conductive substrate; and contacting a matrix to a surface of the biological sample. Further methods optionally include performing mass spectrometry analysis; determining presence of a first analyte in the biological sample; and/or analyzing a second analyte on the surface of the biological sample to determine presence of the first and the second analyte in the biological sample.

### CROSS-REFERENCE TO RELATED APPLICATIONS

Pursuant to 35 U.S.C. § 119 (e), this application is a continuation of International Application PCT/US2023/085163, with an international filing date of Dec. 20, 2023, which claims the benefit of U.S. Provisional Application No. 63/476,532, filed Dec. 21, 2022, the contents of which are incorporated by reference herein in their entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Multi-cellular biological systems display an extraordinary complexity on a multitude of levels. While much of the primary structure of DNA is shared across the trillions of genomes of the cells in the human body, the cells will show a wide diversity in morphology and molecular composition. Currently, there are various techniques for the experimental determination of genomes, transcriptomes, and proteomes, both in tissue and single cells. However, to fully deliver on their potential, these technologies must be matched within the same sample, preferably within the same tissue section, with positional information of the different analytes.

### SUMMARY

Traditionally, bulk methodologies provide an exploratory approach (distinct from more targeted analyses of specific analytes) to investigate the genome, the transcriptome, and/or the proteome data in tissue, resulting in an average view of biomolecules within a biological sample. Single-cell technology has provided the first tools towards a higher level of granularity by providing genome-wide analysis of gene expression as well as open chromatin in individual cells within a tissue. However, the single-cell field for analyzing entire genomes and proteomes is either non-existing or in development due to mainly cost and technical limitations. Importantly, none of the single-cell technologies will provide spatial information since they use FACS sorting of cells/nuclei from dissociated tissue or low-throughput laser capture microdissection of tissue sections.

Spatial technologies are becoming more available with commercial reagents for barcoding gene expression or instruments for mass spectrometry. In an unbiased manner, these platforms allow investigation of (i) gene activity and cell types (e.g., by inference from scRNAseq) and (ii) low molecular compounds, such as neurotransmitters, in a tissue context. However, several technical aspects hinder these analyses from being performed on the same tissue section. For example and without limitation, conductive slides are used in mass spectrometry imaging (MSI), while non-conductive barcoded substrates (e.g., slides) are required for spatial gene expression analysis.

Understanding experimental technologies and the ability to formulate pertinent biological and medical questions must come hand in hand with the design of machine learning algorithms and bioinformatics tools with implementation on high-performance computing hardware. Indeed, multimodal data integration is a current challenge due to noise models and inference between measurement modalities and tissue samples. Thus, a single experimental workflow that allows for a combined collection of biomolecule modalities would provide a significant advantage to the field not only experimentally but also analytically.

Provided herein are methods for analyzing a biological sample, a method comprising: (a) contacting the biological sample with a non-conductive substrate; (b) contacting a matrix to a surface of the biological sample, thereby generating a mass spectrometry sample surface; and (c) performing mass spectrometry analysis of the mass spectrometry sample surface to determine presence of an analyte in the biological sample.

In some embodiments, the non-conductive substrate comprises a plurality of capture probes disposed on a surface of the non-conductive substrate, wherein at least one capture probe of the plurality of capture probes comprises a capture domain and a spatial barcode. In some embodiments, the non-conductive substrate comprises or consists essentially of glass.

In some embodiments, the mass spectrometry analysis comprises laser desorption and ionization and/or electrospray ionization. In some embodiments, the mass spectrometry analysis comprises matrix-assisted laser desorption/ionization-mass spectrometry imaging (MALDI-MSI) or matrix-assisted laser desorption electrospray ionization (MALDESI). In some embodiments, the mass spectrometry analysis further comprises mass spectrometry imaging. In some embodiments, the mass spectrometry analysis is conducted for at least one hour, two hours, three hours, or longer. In some embodiments, the mass spectrometry analysis is performed at room temperature (e.g., about 18-25° C.).

Also provided herein are methods for analyzing a biological sample, a method comprising: (a) contacting the biological sample with a substrate comprising a plurality of capture probes; (b) contacting a matrix to a surface of the biological sample; (c) optionally removing the matrix to provide a further surface of the biological sample; and (d) analyzing an analyte on the further surface of the biological sample to determine presence of the analyte in the biological sample.

In some embodiments, the analyte comprises a RNA, a DNA, or a protein. In some embodiments, the analyte comprises RNA. In some embodiments, at least one capture probe of the plurality of capture probes comprises a capture domain and a spatial barcode. In some embodiments, the analyzing comprises spatial transcriptomics. In some embodiments, the spatial transcriptomics comprises hybridizing the analyte to the capture domain, thereby generating a captured analyte.

Also provided herein are methods for analyzing a biological sample, a method comprising: (a) contacting the biological sample with a substrate comprising a plurality of capture probes, wherein at least one capture probe of the plurality of capture probes comprises a capture domain and a spatial barcode; (b) contacting a matrix to a surface of the biological sample, thereby generating a mass spectrometry sample surface; (c) performing mass spectrometry analysis on the mass spectrometry sample surface to determine presence of a first analyte in the biological sample, thereby providing a further surface of the biological sample after mass spectrometry analysis; and (d) analyzing a second analyte on the further surface of the biological sample to determine presence of the second analyte in the biological sample.

In some embodiments, the mass spectrometry analysis comprises analyzing the first analyte of a plurality of analytes from the mass spectrometry sample surface in a mass spectrometer to determine the presence of the first analyte in the biological sample. In some embodiments, the mass spectrometry analysis further comprises laser desorption and ionization and/or electrospray ionization. In some embodiments, the mass spectrometry analysis comprises matrix-assisted laser desorption/ionization-mass spectrometry imaging (MALDI-MSI) or matrix-assisted laser desorption electrospray ionization (MALDESI). In some embodiments, the mass spectrometry analysis further comprises mass spectrometry imaging. In some embodiments, the mass spectrometry analysis is conducted for at least one hour, two hours, three hours, or longer. In some embodiments, the mass spectrometry analysis is performed at room temperature (e.g., about 18-25° C.).

In some embodiments, the second analyte comprises a RNA, a DNA, or a protein. In some embodiments, the second analyte comprises RNA. In some embodiments, the analyzing comprises spatial transcriptomics.

In some embodiments, the spatial transcriptomics comprises: hybridizing the first analyte or the second analyte (e.g., of a plurality of analytes) to the capture domain, thereby generating a captured analyte; determining (i) all or a part of the sequence of the first analyte or the second analyte, or a complement thereof, and (ii) the spatial barcode, or a complement thereof; and using the determined sequence of (i) and (ii) to analyze the first analyte or the second analyte in the biological sample. In some embodiments, the determining step comprises sequencing. In some embodiments, the analyzing step comprises sequencing the spatial barcode.

In some embodiments, the method further comprises, prior to performing the analyzing step, fixing and/or staining the biological sample. In some embodiments, the fixing comprises methanol fixation. In some embodiments, the staining comprises hematoxylin and/or eosin staining.

In some embodiments, the substrate is a non-conducting substrate. In some embodiments, the analyte or the first analyte is a polymer, a lipid, or a peptide. In some embodiments, the analyte or the second analyte is a DNA molecule, a RNA molecule, a protein, a small molecule, or a metabolite. In some embodiments, the second analyte is mRNA.

In some embodiments, the contacting the matrix in step (b) comprises providing the matrix within a solvent. In some embodiments, the method further comprises, after or during step (b), rinsing the mass spectrometry sample surface with a further solvent. In some embodiments, the matrix is selected from a group consisting of: 9-aminoacridine (9-AA), 2,5-dihydroxybenzoic acid (DHB), norharmane, and 2-fluoro-1-methyl pyridinium (FMP-10), or a combination thereof.

In some embodiments, the substrate comprises or is a glass slide. In some embodiments, the substrate comprises or is a gene expression array. In some embodiments, the capture domain comprises a poly(T) sequence. In some embodiments, the biological sample is a tissue sample. In some embodiments, the tissue sample is a fresh frozen tissue section.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4A shows an exemplary schematic of a method, where a tissue sample (e.g., a non-embedded snap-frozen tissue sample) is sectioned and mounted on a substrate (e.g., a non-conductive, barcoded Visium gene expression array); the tissue section is contacted with a matrix (e.g., a MALDI-MSI matrix); and mass spectrometry (MS) or MS imaging (MSI) is performed on the matrix coated tissue section. Optionally, further analysis (e.g., spatial transcriptomics) is performed on the tissue section. FIG. 4B shows an exemplary flowchart of the method described in FIG. 4A.

FIG. 5A shows an exemplary schematic of a method, where a tissue sample (e.g., a non-embedded snap-frozen tissue sample) is sectioned and mounted on a substrate (e.g., a non-conductive, barcoded Visium gene expression array); the tissue section is contacted with a matrix (e.g., a MALDI-MSI matrix); the matrix is optionally removed from the tissue section; and further analysis (e.g., spatial transcriptomics) is performed on the tissue section. FIG. 5B shows an exemplary flowchart of the method described in FIG. 5A.

FIGS. 6A-6E show that Spatial Multimodal Analysis (SMA) is feasible, efficient, and highly reproducible. FIG. 6A shows an exemplary diagram of the method, where non-embedded snap-frozen tissue samples are sectioned and mounted on non-conductive, barcoded Visium gene expression arrays; tissue sections placed on non-conductive slides (indicated as gray rectangles with a black outline) and conductive slides (indicated as gray rectangles with a jagged arrow symbol) were used for comparison with standard Visium and MSI protocols; tissue sections are contacted with several MALDI-MSI matrices, while some tissue sections lacked a matrix as an internal control; MSI is performed on matrix coated tissue sections; tissue sections are Hematoxylin and Eosin stained and imaged with light microscopy; and spatial transcriptomics is performed on all tissue sections. FIG. 6B shows pairwise gene to gene detection rates and molecule to molecule correlations in biological and technical replicates. FIG. 6C shows UMAP of SMA ST barcoded features colored by individual tissue sections (upper), MALDI matrices (middle), and Seurat clusters (lower). FIG. 6D shows percentage of detected transcripts across biological and technical replicates. FIG. 6E shows spatial mapping of mouse brain tissue sections (striatal level, 0.49 mm from bregma) showing Seurat clusters of transcripts for tissue sections contacted with one of three different MALDI matrices (FMP-10, 9AA, DHB) and one sample processed with a standard Visium gene expression protocol (i-CTRL).

FIGS. 7A-7D show Spatial Multimodal Analysis (SMA) application to a mouse model and a human postmortem brain having Parkinson's disease (PD). FIG. 7A shows a cartoon of a mouse brain showing a sagittal section (left) indicating the depth (0.49 and −3.39 mm, distance from bregma) of the coronal sections for striatum and substantia nigra. The coronal tissue section (right) shows the striatal regions of the two hemispheres and is illustrating the DA depleted striatum (red) induced by unilateral 6-OHDA lesion. FIGS. 7B-7C show representative sections from substantia nigra (a) and striatum (b) of the mouse Parkinson's Disease model (FIG. 7B) and human brain (FIG. 7C). From left to right: HE staining, clustering of transcriptomics data (mRNA), dopamine expression, combine module score of genes upregulated in the intact hemisphere (mouse samples) or brain area (human sample). FIG. 7D shows, from left to right: top 20 differentially upregulated and downregulated genes in the intact hemispheres of mouse substantia nigra, mouse striatum, and human intact area of the striatum.

FIG. 8 shows SMA using four different MALDI matrices on non-conductive substrates. Panel a) shows tissue sections placed on Visium Tissue Optimization slides and spray contacted with four different MALDI matrices. Dashed areas show areas of interest imaged with MSI (see, panel b)). Panel b) shows representative MSI results: i) C-18 L-Carnitine; ii) 867-5682 Da peak; iii) ADP; iv) GABA. Nor+ and Nor−: Norharmane analyzed in positive and negative mode. Panel c) shows fluorescence microscopy images of RNA captured with poly-dT probes and retrotranscribed using fluorescent nucleotides after MSI.

FIGS. 9A-9B show electropherograms of SMA processed samples. FIG. 9A shows electropherograms of 6 tissue sections cut from the same tissue sample and processed with 3 different MALDI matrices and SMA. FIG. 9B shows electropherograms of 4 tissue sections cut from 3 different samples and processed with FMP10 and SMA. iCTRL: Visium internal control.

FIG. 10 show spatial distribution of detected genes from tissue sections contacted with MALDI matrices. Non-filtered Visium data from all experiments in the dataset were used to plot gene counts for all the barcoded features with counts higher than zero.

FIG. 11 shows pairwise scatterplots of gene to gene detection rates and corresponding gene to gene detection rate correlations of technical replicates. For this analysis, consecutive striatum sections of the same mouse (mPD3) were used. ***: p-val lower than 0.0001.

FIG. 12 shows pairwise scatterplots of gene to gene detection rates and corresponding gene to gene detection rate correlations of biological replicates. For this analysis, several striatum sections of 3 different 6-OHDA mice (mPD1, mPD3, mPD4) were used. ***: p-val lower than 0.0001.

FIG. 13 shows an UpSet plot of detected transcripts across technical replicates. The top-right barplot represents the total number of RNA molecules detected across the conditions illustrated below the barplot. For each bar, only the conditions highlighted by a black dot in the lower panel are taken into account. The low-left panel represents the total number of molecules detected under each condition. For this analysis, the same striatum sections of the correlation analysis were used.

FIG. 14 shows an UpSet plot of detected transcripts across biological replicates. The top-right barplot represents the total number of RNA molecules detected across the conditions illustrated below the barplot. For each bar, only the conditions highlighted by a black dot in the lower panel are taken into account. The low-left panel represents the total number of molecules detected under each condition. For this analysis, the same striatum sections of the correlation analysis were used.

FIG. 15 shows a selection of mass spectrometry ion images from a mouse brain tissue section detecting neurotransmitters and metabolites, including a) GABA-H2O+FMP-10; b) Taurine+FMP-10; c) Serotonin+FMP-10; d) Histidine+FMP-10; e) 3-MT+FMP-10; f) Dopamine+FMP-10; g) Dopamine+2FMP-10; h) DOPAL+FMP-10; i) DOPAC+2FMP-10; j) Norepinephrine+2FMP-10; k) Tocopherol+FMP-10; and 1) Scanned tissue.

FIG. 16 shows a scanned image of the Human Brain tissue on a Visium glass slide. FIG. 16A shows a whole tissue scan with annotated brain regions, where double-lined squares indicate individual spatial arrays. FIG. 16B shows ion images of dopamine. FIG. 16C shows ion images of 3-MT. FIG. 16D shows ion images of serotonin. FIG. 16E shows ion images of norepinephrine (double derivatized). All ion distributions are scaled to 50% of the maximum intensity and are all displayed as single derivatized species.

### DETAILED DESCRIPTION

### I. Introduction

Multi-cellular biological systems display an extraordinary complexity on a multitude of levels. While much of the genome's primary structure is shared across the trillions of cells in the human body, tissues show great diversity in morphology and cellular composition. Complete profiling of the cellular and molecular networks is needed to fully understand the biological mechanisms that lead to such diversity.

Spatial transcriptomics (ST) allows the measurement of both genome-wide mRNA expression and positional information of the mRNA in a tissue sample. While aspects of ST technologies such as field of view, cellular resolution, target content, and sensitivity vary, they allow for the compilation of a gene-expression count table with tissue coordinates.

Mass spectrometry imaging (MSI) is a technology that enables label-free measurement of the abundance and molecular distribution of lipids, peptides, proteins, along with drugs and their metabolites directly in fresh frozen tissue sections. Using matrix-assisted laser desorption/ionization (MALDI)-MSI, a matrix (a small organic molecule) is applied onto the surface of tissue sections mounted on a glass slide. Focusing a pulsed laser beam onto the tissue section generates ionic species from components of the tissue section. An ordered array of mass spectra is acquired at defined raster positions allowing for the collection of mass-to-charge (m/z) spectra in two-dimensions across the tissue section. The resulting ions in the spectra are identified by tandem MS (MS/MS) directly in tissue sections and/or mass-matched towards reference molecules. The imaging process can take hours to days, depending on the size of the imaged tissue area and the selected lateral resolution.

Spatial gene expression and mass spectrometry technologies are becoming more established in spatial biology. However, they are currently applied in separate experiments due to experimental constraints such as the usage of non-conductive (Spatial Transcriptomic) vs. conductive (MALDI-MSI) slides, or RNA degradation that can arise from exposing the tissue section to laser ablation and lengthy (e.g., hourly) imaging sessions. As described herein, a multimodal spatial approach, “spatial multimodal analysis (SMA)”, can expand the capabilities of current spatial assays to measure analytes (e.g., metabolites) and gene expression simultaneously. The spatial multimodal analysis methods described herein can combine histology, mass spectrometry imaging, and spatial transcriptomics within a single tissue section, enabling parallel analysis of tissue morphology, transcripts, and small molecules, with retained specificity and sensitivity.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; P.C.T. Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of P.C.T. Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of P.C.T. Publication No. WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). In some instances, the biological sample is fixed using PAXgene. PAXgene is a formalin-free, non-cross-linking fixative that preserves morphology and biomolecules. It is a mixture of different alcohols, acid, and a soluble organic compound. Ergin B. et al., J Proteome Res. 2010 Oct. 1; 9 (10): 5188-96 appears to have first developed and described PAXgene. Kap M. et al., PLOS One.; 6 (11): e27704 (2011) and Mathieson W. et al., Am J Clin Pathol.; 146 (1): 25-40 (2016) both describe and evaluate PAXgene for tissue fixation. Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of P.C.T. Publication No. WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of P.C.T. Publication No. WO 2020/176788 and/or U.S. Patent Application Publication No. 2020/0277663.

Array-based spatial analysis methods often involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can further include a cleavage domain and/or a functional domain (e.g., a primer-binding site, such as for next-generation sequencing (NGS)). See, e.g., Section (II)(b) (e.g., subsections (i)-(vi)) of P.C.T. Publication No. WO 2020/176788 and/or U.S. Patent Application Publication No. 2020/0277663. Generation of capture probes can be achieved by any appropriate method, including those described in Section (II)(d)(ii) of P.C.T. Publication No. WO 2020/176788 and/or U.S. Patent Application Publication No. 2020/0277663.

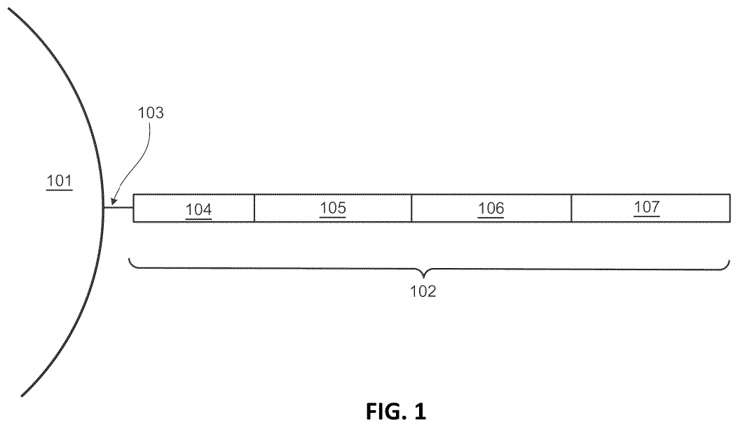

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

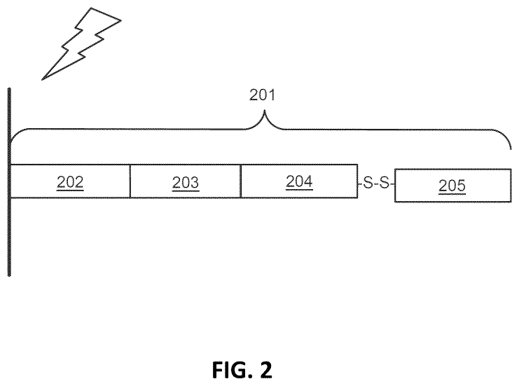

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

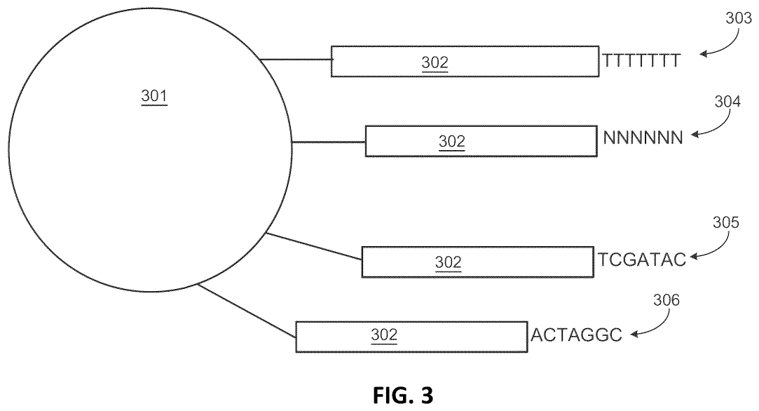

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); and/or (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of P.C.T. Publication No. WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of P.C.T. Publication No. WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of P.C.T. Publication No. WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of P.C.T. Publication No. WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of P.C.T. Publication No. WO 2020/176788 and/or U.S. Patent Application Publication No. 2020/0277663. Some quality control measures are described in Section (II)(h) of P.C.T. Publication No. WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and/or 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of P.C.T. Publication No. WO 2020/176788 and/or U.S. Patent Application Publication No. 2020/0277663. Exemplary features and geometric attributes of an array can be found in Sections (II)(d)(i), (II)(d)(iii), and (II)(d)(iv) of P.C.T. Publication No. WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described for example in Section (II)(e) of P.C.T. Publication No. WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of P.C.T. Publication No. WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45 (14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a homopolymer sequence (e.g., a poly(A) sequence)). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored and retrieved as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of P.C.T. Publication No. WO2020/176788 and/or U.S. Patent Application Publication No. 2020/0277663. See, for example, the Exemplary embodiment starting with “In some non-limiting examples of the workflows described herein, the sample can be immersed . . . ” of P.C.T. Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II)(e)(ii) and/or (V) of P.C.T. Publication No. WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of P.C.T. Publication No. WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in P.C.T. Publication No. WO 2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in P.C.T. Publication No. 2020/053655 and spatial analysis methods are generally described in P.C.T. Publication No. WO 2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of P.C.T. Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods for Analysis of Molecules and Spatial Gene Expression

Provided herein are methods for analyzing a biological sample that include (a) contacting the biological sample with a substrate (e.g., a non-conductive substrate); (b) contacting a matrix to a surface of the biological sample, thereby generating a mass spectrometry sample surface; and (c) performing mass spectrometry analysis of the mass spectrometry sample surface to determine presence of an analyte in the biological sample.

Also provided herein are methods for analyzing a biological sample that include (a) contacting the biological sample with a substrate (e.g., a substrate comprising a plurality of capture probes, wherein at least one capture probe of the plurality of capture probes comprises a capture domain and a spatial barcode); (b) contacting a matrix to a surface of the biological sample; (c) optionally removing the matrix to provide a further surface of the biological sample; and (d) analyzing an analyte on the further surface of the biological sample to determine presence of the analyte in the biological sample.

Also provided herein are methods for analyzing a biological sample that include (a) contacting the biological sample with a substrate (e.g., a substrate comprising a plurality of capture probes, wherein at least one capture probe of the plurality of capture probes comprises a capture domain and a spatial barcode); (b) contacting a matrix to a surface of the biological sample, thereby generating a mass spectrometry sample surface; (c) performing mass spectrometry analysis on the mass spectrometry sample surface to determine presence of a first analyte in the biological sample, thereby providing a further surface of the biological sample after mass spectrometry analysis; and (d) analyzing a second analyte on the further surface of the biological sample to determine presence of the second analyte in the biological sample. Additional details regarding these methods are described herein.

### (A) Exemplary Biological Samples

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section. In some embodiments, the tissue is flash-frozen and sectioned. Any suitable methods described herein or known in the art can be used to flash-freeze and section the tissue sample. In some embodiments, the biological sample, e.g., the tissue, is flash-frozen using liquid nitrogen before sectioning. In some embodiments, the sectioning is performed using cryosectioning. In some embodiments, the methods further comprise a thawing step, after the cryosectioning. In some embodiments, the tissue sample is a fresh frozen tissue section.

In some embodiments, the biological sample, e.g., the tissue sample is fixed, for example in methanol, acetone, paraformaldehyde (PFA) or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probe oligonucleotides that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to deaminating the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some embodiments, the sample is a human sample. In some embodiments, the sample is a human brain tissue sample. In some embodiments, the sample is a non-human sample. In some embodiments, the tissue is a patient derived organoid. In some embodiments, the sample is a mouse brain sample.

### (B) Exemplary Substrates

In some instances, the biological sample is placed (e.g., mounted or otherwise immobilized) on a substrate. The substrate can be any solid or semi-solid support upon which a biological sample can be mounted. In some embodiments, the substrate is a slide. In some embodiments, the slide is a glass slide. In some embodiments, the substrate is made of glass, silicon, paper, hydrogel, polymer monoliths, or other material known in the art. In some embodiments, the substrate is comprised of an inert material or matrix (e.g., glass slides) that has been functionalized by, for example, treating the substrate with a material comprising reactive groups which facilitate mounting of the biological sample.

As used herein, a substrate can generally have any suitable form or format. For example, a substrate can be flat, curved, e.g., convexly or concavely curved. For example, a substrate can be curved towards the area where the interaction between a biological sample, e.g., tissue sample, and a substrate takes place. In some embodiments, a substrate is flat, e.g., planar, such as a planar chip or slide. A substrate can contain one or more patterned surfaces within the substrate (e.g., channels, wells, projections, ridges, divots, etc.).

In some embodiments, a substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments wherein a substrate structure is flat, the substrate structure can be any appropriate type of support having a flat surface (e.g., a chip or a slide such as a microscope slide).

In some embodiments, substrates can optionally include various structures such as, but not limited to, projections, ridges, and channels. A substrate can be micropatterned to limit lateral diffusion of analytes (e.g., to improve resolution of spatial analysis). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a substrate is modified to contain one or more wells, using techniques such as (but not limited to) stamping, microetching, or molding techniques. In some embodiments in which a substrate includes one or more wells, the substrate can be a concavity slide or cavity slide. For example, wells can be formed by one or more shallow depressions on the surface of the substrate. In some embodiments, where a substrate includes one or more wells, the wells can be formed by attaching a cassette (e.g., a cassette containing one or more chambers) to a surface of the substrate structure.

In some embodiments where the substrate is modified to contain one or more structures, including but not limited to, wells, projections, ridges, or markings, the structures can include physically altered sites. For example, a substrate modified with various structures can include physical properties, including, but not limited to, physical configurations, magnetic or compressive forces, chemically functionalized sites, chemically altered sites, and/or electrostatically altered sites. In some embodiments where the substrate is modified to contain various structures, including but not limited to wells, projections, ridges, or markings, the structures can be applied in a pattern. Alternatively, the structures can be randomly distributed.

In some embodiments, a substrate includes one or more markings on its surface. For example, the substrate can include a sample area indicator identifying the sample area. In some embodiments, the substrate can include a fiducial mark. In some embodiments, the substrate does not comprise a fiducial mark. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007, the entire contents of which are incorporated herein by reference. In some embodiments, a fiducial marker can be a physical particle (e.g., a nanoparticle, a microsphere, a nanosphere, a bead, a post, or any of the other exemplary physical particles described herein or known in the art).

A wide variety of substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

In some embodiments, the substrate comprises a plurality (e.g., array) of capture probes, each comprising a spatial barcode. In some embodiments, the substrate comprises an array, wherein the array comprises a plurality of features collectively positioned on the substrate. In other embodiments, the substrate can include a non-conductive substrate (e.g., any described herein).

### Arrays for Analyte Capture

In some embodiments, an array can include a capture probe attached directly or indirectly to the substrate. The capture probe can include a capture domain (e.g., a nucleotide sequence) that can specifically bind (e.g., hybridize) to a target analyte (e.g., mRNA, DNA, or protein) within a sample. In some embodiments, the binding of the capture probe to the target analyte (e.g., hybridization) can be detected and quantified by detection of a visual signal, e.g., a fluorophore, a heavy metal (e.g., silver ion), or chemiluminescent label, which has been incorporated into the target analyte. In some embodiments, the intensity of the visual signal correlates with the relative abundance of each analyte in the biological sample. Since an array can contain thousands or millions of capture probes (or more), an array can interrogate many analytes in parallel and/or in series.

In some embodiments, a substrate includes one or more capture probes that are designed to capture analytes from one or more organisms. In a non-limiting example, a substrate can contain one or more capture probes designed to capture mRNA from one organism (e.g., a human) and one or more capture probes designed to capture DNA from a second organism (e.g., a bacterium or virus).

The capture probes can be attached to a substrate or feature using a variety of techniques. In some embodiments, the capture probe is directly attached to a feature that is fixed on an array. In some embodiments, the capture probes are immobilized to a substrate by chemical immobilization. For example, a chemical immobilization can take place between functional groups on the substrate and corresponding functional elements on the capture probes. Exemplary corresponding functional elements in the capture probes can either be an inherent chemical group of the capture probe, e.g., a hydroxyl group, or a functional element can be introduced on to the capture probe. An example of a functional group on the substrate is an amine group. In some embodiments, the capture probe to be immobilized includes a functional amine group or is chemically modified in order to include a functional amine group.

In some embodiments, the capture probe is a nucleic acid. In some embodiments, the capture probe is immobilized on a substrate or feature via its 5′ end. In some embodiments, the capture probe is immobilized on a substrate or feature via its 5′ end and includes from the 5′ to 3′ end: one or more barcodes (e.g., a spatial barcode and/or a UMI) and one or more capture domains. In some embodiments, the capture probe is immobilized on a substrate or feature via its 5′ end and includes from the 5′ to 3′ end: one barcode (e.g., a spatial barcode and/or a UMI) and one capture domain. In some embodiments, the capture probe is immobilized on a substrate or feature via its 5′ end and includes from the 5′ to 3′ end: a cleavage domain, a functional domain, one or more barcodes (e.g., a spatial barcode and/or a UMI), and a capture domain.

In some embodiments, the capture probe is immobilized on a substrate or feature via its 5′ end and includes from the 5′ to 3′ end: a cleavage domain, a functional domain, one or more barcodes (e.g., a spatial barcode and/or a UMI), a second functional domain, and a capture domain. In some embodiments, the capture probe is immobilized on a substrate or feature via its 5′ end and includes from the 5′ to 3′ end: a cleavage domain, a functional domain, a spatial barcode, a UMI, and a capture domain. In some embodiments, the capture probe is immobilized on a substrate or feature via its 5′ end and does not include a spatial barcode. In some embodiments, the capture probe is immobilized on a substrate or feature via its 5′ end and does not include a UMI. In some embodiments, the capture probe includes a sequence (e.g., a primer binding site) for initiating a sequencing reaction.

In some embodiments, the capture probe is immobilized on a substrate or feature via its 3′ end. In some embodiments, the capture probe is immobilized on a substrate or feature via its 3′ end and includes from the 3′ to 5′ end: one or more barcodes (e.g., a spatial barcode and/or a UMI) and one or more capture domains. In some embodiments, the capture probe is immobilized on a substrate or feature via its 3′ end and includes from the 3′ to 5′ end: one barcode (e.g., a spatial barcode or a UMI) and one capture domain. In some embodiments, the capture probe is immobilized on a substrate or feature via its 3′ end and includes from the 3′ to 5′ end: a cleavage domain, a functional domain, one or more barcodes (e.g., a spatial barcode and/or a UMI), and a capture domain. In some embodiments, the capture probe is immobilized on a substrate or feature via its 3′ end and includes from the 3′ to 5′ end: a cleavage domain, a functional domain, a spatial barcode, a UMI, and a capture domain.

The localization of the functional group within the capture probe to be immobilized can be used to control and shape the binding behavior and/or orientation of the capture probe, e.g., the functional group can be placed at the 5′ or 3′ end of the capture probe or within the sequence of the capture probe. In some embodiments, a capture probe can further include a substrate. A typical substrate for a capture probe to be immobilized includes moieties which are capable of binding to such capture probes, e.g., to amine-functionalized nucleic acids. Examples of such substrates are carboxy, aldehyde, or epoxy substrates.

In some embodiments, the substrates on which capture probes can be immobilized can be chemically activated, e.g., by the activation of functional groups available on the substrate. The term “activated substrate” relates to a material in which interacting or reactive chemical functional groups are established or enabled by chemical modification procedures. For example, a substrate including carboxyl groups can be activated before use. Furthermore, certain substrates contain functional groups that can react with specific moieties already present in the capture probes.

In some embodiments, a covalent linkage is used to directly couple a capture probe to a substrate. In some embodiments, a capture probe is indirectly coupled to a substrate through a linker separating the “first” nucleotide of the capture probe from the substrate, e.g., a chemical linker. In some embodiments, a capture probe does not bind directly to the substrate, but interacts indirectly, for example by binding to a molecule which itself binds directly or indirectly to the substrate. In some embodiments, the capture probe is indirectly attached to a substrate (e.g., attached to a substrate via a solution including a polymer).

In some embodiments where the capture probe is immobilized on a feature of the array indirectly, e.g., via hybridization to a surface probe capable of binding the capture probe, the capture probe can further include an upstream sequence (5′ to the sequence that hybridizes to the nucleic acid, e.g., RNA of the tissue sample) that is capable of hybridizing to 5′ end of a surface probe. Alone, the capture domain of the capture probe can be seen as a capture domain oligonucleotide, which can be used in the synthesis of the capture probe in embodiments where the capture probe is immobilized on the array indirectly.

In some embodiments, a substrate is comprised of an inert material or matrix (e.g., glass slides) that has been functionalized by, for example, treating the substrate with a material comprising reactive groups which enable immobilization of capture probes. See, for example, WO 2017/019456, the entire contents of which is herein incorporated by reference. Non-limiting examples include polyacrylamide hydrogels supported on an inert substrate (e.g., glass slide; see WO 2005/065814 and U.S. Patent Application No. 2008/0280773, the entire contents of which is incorporated herein by reference).

In some embodiments, capture probes are immobilized on a functionalized substrate using covalent methods. Methods for covalent attachment include, for example, condensation of amines and activated carboxylic esters (e.g., N-hydroxysuccinimide esters); condensation of amine and aldehydes under reductive amination conditions; and cycloaddition reactions such as the Diels-Alder [4+2] reaction, 1,3-dipolar cycloaddition reactions, and [2+2] cycloaddition reactions. Methods for covalent attachment also include, for example, click chemistry reactions, including [3+2] cycloaddition reactions (e.g., Huisgen 1,3-dipolar cycloaddition reaction and copper (I)-catalyzed azide-alkyne cycloaddition (CuAAC)); thiol-ene reactions; the Diels-Alder reaction and inverse electron demand Diels-Alder reaction; [4+1] cycloaddition of isonitriles and tetrazines; and nucleophilic ring-opening of small carbocycles (e.g., epoxide opening with amino oligonucleotides). Methods for covalent attachment also include, for example, maleimides and thiols; and para-nitrophenyl ester-functionalized oligonucleotides and polylysine-functionalized substrate. Methods for covalent attachment also include, for example, disulfide reactions; radical reactions (see, e.g., U.S. Pat. No. 5,919,626, the entire contents of which are herein incorporated by reference); and hydrazide-functionalized substrate (e.g., wherein the hydrazide functional group is directly or indirectly attached to the substrate) and aldehyde-functionalized oligonucleotides (see, e.g., Yershov et al. (1996) Proc. Natl. Acad. Sci. USA 93, 4913-4918, the entire contents of which are herein incorporated by reference).

In some embodiments, capture probes are immobilized on a functionalized substrate using photochemical covalent methods. Methods for photochemical covalent attachment include, for example, immobilization of antraquinone-conjugated oligonucleotides (see, e.g., Koch et al. (2000) Bioconjugate Chem. 11, 474-483, the entire contents of which is herein incorporated by reference).

In some embodiments, capture probes are immobilized on a functionalized substrate using non-covalent methods. Methods for non-covalent attachment include, for example, biotin-functionalized oligonucleotides and streptavidin-treated substrates (see, e.g., Holmstrøm et al. (1993) Analytical Biochemistry 209, 278-283 and Gilles et al. (1999) Nature Biotechnology 17, 365-370, the entire contents of which are herein incorporated by reference).

In some embodiments, an oligonucleotide (e.g., a capture probe) can be attached to a substrate or feature according to the methods set forth in U.S. Pat. Nos. 6,737,236, 7,259,258, 7,375,234, 7,427,678, 5,610,287, 5,807,522, 5,837,860, and 5,472,881; U.S. Patent Application Publication Nos. 2008/0280773 and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents is incorporated herein by reference.

### (C) Matrix Application and Removal

In some embodiments, a matrix can be applied to a biological sample (e.g., tissue section) contacted on a substrate (e.g., microscope slide). In some embodiments, the matrix can be applied manually or automatically. In some embodiments, the matrix absorbs at the laser wavelength and ionizes the analyte. Matrix selection and solvent system can rely upon the type of analyte desired in imaging, wherein the analyte must be soluble in the solvent in order to mix and recrystallize the matrix. In some embodiments, the matrix can have a homogeneous coating in order to increase sensitivity, intensity, and reproducibility. In some embodiments, minimal solvent can be used when applying the matrix in order to avoid delocalization.

In some embodiments, the matrix can be applied by spraying, wherein the matrix is sprayed as very small droplets, onto the surface of the sample, allowed to dry, and re-coated until there is enough matrix to analyze the biological sample. In some embodiments, the size of the crystals depend on the solvent system used. Any useful coating methods can be employed to apply the matrix, such as dried-droplet crystallization, vacuum-drying crystallization, crushed crystal methods, fast-evaporation methods, sandwich methods, spin-coating, electrospraying, airbrushing, spray-coating, spotting, sublimation, as well as variations of any of these.

In some embodiments, the matrix can be applied by using sublimation to make uniform matrix coatings with very small crystals. The matrix is placed in a sublimation chamber with the biological sample mounted on the substrate inverted above it, where heat is applied to the matrix, causing it to sublime and condense onto the surface of the biological sample. In some embodiments, controlling the heating time controls the thickness of the matrix on the biological sample and the size of the crystals formed.

In some embodiments, the matrix is selected from a group consisting of: 9-aminoacridine (9-AA), 2,5-dihydroxybenzoic acid (DHB), norharmane, and 2-fluoro-1-methyl pyridinium (FMP-10), or a combination thereof, as well as salts thereof. In some embodiments, a matrix can include 9-Aminoacridine (9-AA), 2,5-dihydroxybenzoic acid (DHB), Norharmane, or 2-fluoro-1-methyl pyridinium (FMP-10). Other non-limiting examples of matrices include one or more of the following: 2,5-dihydroxybenzoic acid (DHB); 2-hydroxy-5-methoxybenzoic acid; 2,5-dihydroxyterephthalic acid; 1,4-dihydroxy-2-naphthoic acid; 3,7-dihydroxy-2-naphthoic acid; 2-(4-hydroxyphenylazo)benzoic acid (HABA); 3,5-dimethoxy-4-hydroxycinnamic acid (or sinapinic acid, SA); 4-hydroxy-3-methoxycinnamic acid (or ferulic acid); α-cyano-4-hydroxycinnamic acid (CHCA or CCA); 4-chloro-α-cyanocinnamic acid (CI-CCA); 3,4-dihydroxycinnamic acid (or caffeic acid); picolinic acid (PA); 3-hydroxypicolinic acid (3-HPA); anthranilamide; 2-(2-aminoethylamino)-5-nitropyridine; 7-hydroxycoumarin; 7-hydroxycoumarin acetic acid; 4-hydroxycoumarin; 6,7-dihydroxycoumarin; 7,8-dihydroxy-6-methoxycoumarin; 2-mercaptobenzothiazole (MBT); 4-nitroaniline; dithranol (DTN); 1,6-diphenyl-1,3,5-hexatriene; 9-aminoacridine (9-AA); 1,5-diaminonapthalene (DAN); 1,8-bis(dimethylamino) naphthalene; 2,5-dihydroxyacetophenone (DHA); 2,6-dihydroxyacetophenone (DHAP); 2,4,6-trihydroxyacetophenone (THAP); harmane; norharmane; harmine; 4-aminoquinaldine; curcumin; trans-2-[3-(4-tert-butylphenyl)-2-methyl-2-propenylidene]malononitrile (DCTB); 2-fluoro-1-methyl pyridinium (FMP-10); and the like, as well as combinations or mixtures including any of these (e.g., a mixture of DHB with 2-hydroxy-5-methoxybenzoic acid, CHCA, 6-aza-2-thiothymine, 2,4-dinitrobenzoic acid, and the like) and salts or solvates of any of these (e.g., including sodium salts, hydrochloride salts, lithium salts, hydrates, and the like).

Yet other non-limiting examples of matrices include one or more of the following: benzoic acid, hydroxybenzoic acid, dihydroxybenzoic acid, terephthalic acid, naphthoic acid, cinnamic acid, hydroxycinnamic acid, picolinic acid, benzamide, aniline, acridine, quinoline, naphthalene, anthracene, acetophenone, pyridine, coumarin, norharmane, as well as modified forms of any of these (e.g., modified forms having one or more hydroxyl, methoxy, ethoxy, methyl, ethyl, halo, amino, dialkylamino, nitro, and/or cyano groups), or a salt of any of these.

In some embodiments, contacting the matrix to the biological sample comprises providing the matrix within a solvent. In some embodiments, contacting the matrix to the biological sample can further include, after or during the contacting, rinsing the mass spectrometry sample surface with a further solvent. In some embodiments, the solvent comprises acetonitrile, methanol, ethanol, propanol, water, acetone, chloroform, or acetonitrile mixed with (trifluoroacetic acid) TFA, as well as combinations thereof. In some embodiments, a matrix comprising FMP-10 can be provided within a solvent comprising acetonitrile. In some embodiments, a matrix comprising 9-AA can be provided within a solvent comprising methanol. In some embodiments, a matrix comprising DHB can be provided within a solvent comprising a mix of acetonitrile and TFA.

In some embodiments, the substrate is a conductive substrate. In some embodiments, the conductive substrate is a conductive microscope slide. In some embodiments, the conductive substrate is a metal plate. In some embodiments, the substrate is a non-conductive substrate. In some embodiments, the non-conductive substrate comprises or consists essentially of glass. In some embodiments, the non-conductive substrate is a glass slide. In other embodiments, the non-conductive substrate does not include a metal (e.g., a transition metal or a post-transition metal), a metal oxide, a metal alloy (e.g., an alloy including a metal), or a doped form thereof (e.g., a doped metal oxide, such as indium tin oxide).

In some embodiments, the non-conductive substrate is a gene expression array. In some embodiments, the non-conductive substrate comprises a plurality of capture probes disposed on a surface of the non-conductive substrate, and wherein at least one capture probe of the plurality of capture probes comprises a capture domain and a spatial barcode. In some embodiments, the capture domain can include a poly(T) sequence. In some embodiments, the non-conductive substrate comprises a resistivity from about 10⁻⁶ to about 10¹⁶ Ω·m (determined at about 20° C.), including ranges therebetween (e.g., 10⁻⁶ to 1 Ω·m, 10⁻⁶ to 10 Ω·m, 10⁻⁶ to 10² Ω·m, 10⁻⁶ to 10³ Ω·m, 10⁻⁶ to 10⁴ Ω·m, 10⁻⁶ to 10⁵ Ω·m, 10⁻⁶ to 10⁶ Ω·m, 10⁻⁶ to 10⁷ Ω·m, 10⁻⁶ to 10⁸ Ω·m, 10⁻⁶ to 10⁹ Ω·m, 10⁻⁶ to 10¹⁰ Ω·m, 10⁻⁶ to 10¹¹ Ω·m, 10⁻⁶ to 10¹² Ω·m, 10⁻⁶ to 10¹³ Ω·m, 10⁻⁶ to 10¹⁴ Ω·m, 10⁻⁶ to 10¹⁵ Ω·m, 10⁻⁵ to 1 Ω·m, 10⁻⁵ to 10 Ω·m, 10⁻⁵ to 10² Ω·m, 10⁻⁵ to 10³ Ω·m, 10⁻⁵ to 10⁴ Ω·m, 10⁻⁵ to 10⁵ Ω·m, 10⁻⁵ to 10⁶ Ω·m, 10⁻⁵ to 10⁷ Ω·m, 10⁻⁵ to 10⁸ Ω·m, 10⁻⁵ to 10⁹ Ω·m, 10⁻⁵ to 10¹⁰ Ω·m, 10⁻⁵ to 10¹¹ Ω·m, 10⁻⁵ to 10¹² Ω·m, 10⁻⁵ to 10¹³ Ω·m, 10⁻⁵ to 10¹⁴ Ω·m, 10⁻⁵ to 10¹⁵ Ω·m, 10⁻⁵ to 10¹⁶ Ω·m, 10⁻⁴ to 1 Ω·m, 10⁻⁴ to 10 Ω·m, 10⁻⁴ to 10² Ω·m, 10⁻⁴ to 10³ Ω·m, 10⁻⁴ to 10⁴ Ω·m, 10⁻⁴ to 10⁵ Ω·m, 10⁻⁴ to 10⁶ Ω·m, 10⁻⁴ to 10⁷ Ω·m, 10⁻⁴ to 10⁸ Ω·m, 10⁻⁴ to 10⁹ Ω·m, 10⁻⁴ to 10¹⁰ Ω·m, 10⁻⁴ to 10¹¹ Ω·m, 10⁻⁴ to 10¹² Ω·m, 10⁻⁴ to 10¹³ Ω·m, 10⁻⁴ to 10¹⁴ Ω·m, 10⁻⁴ to 10¹⁵ Ω·m, 10⁻⁴ to 10¹⁶ Ω·m, 10⁻³ to 1 Ω·m, 10⁻³ to 10 Ω·m, 10⁻³ to 10² Ω·m, 10⁻³ to 10³ Ω·m, 10⁻³ to 10⁴ Ω·m, 10⁻³ to 10⁵ Ω·m, 10⁻³ to 10⁶ Ω·m, 10⁻³ to 10⁷ Ω·m, 10⁻³ to 10⁸ Ω·m, 10⁻³ to 10⁹ Ω·m, 10⁻³ to 10¹⁰ Ω·m, 10⁻³ to 10¹¹ Ω·m, 10⁻³ to 10¹² Ω·m, 10⁻³ to 10¹³ Ω·m, 10⁻³ to 10¹⁴ Ω·m, 10⁻³ to 10¹⁵ Ω·m, 10⁻³ to 10¹⁶ Ω·m, 10⁻² to 1 Ω·m, 10⁻² to 10 Ω·m, 10⁻² to 10² Ω·m, 10⁻² to 10³ Ω·m, 10⁻² to 10⁴ Ω·m, 10⁻² to 10⁵ Ω·m, 10⁻² to 10⁶ Ω·m, 10⁻² to 10⁷ Ω·m, 10⁻² to 10⁸ Ω·m, 10⁻² to 10⁹ Ω·m, 10⁻² to 10¹⁰ Ω·m, 10⁻² to 10¹¹ Ω·m, 10⁻² to 10¹² Ω·m, 10⁻² to 10¹³ Ω·m, 10⁻² to 10¹⁴ Ω·m, 10⁻² to 10¹⁵ Ω·m, 10⁻² to 10¹⁶ Ω·m, 10⁻¹ to 1 Ω·m, 10⁻¹ to 10 Ω·m, 10⁻¹ to 10² Ω·m, 10⁻¹ to 10³ Ω·m, 10⁻¹ to 10⁴ Ω·m, 10⁻¹ to 10⁵ Ω·m, 10⁻¹ to 10⁶ Ω·m, 10⁻¹ to 10⁷ Ω·m, 10⁻¹ to 10⁸ Ω·m, 10⁻¹ to 10⁹ Ω·m, 10⁻¹ to 10¹⁰ Ω·m, 10⁻¹ to 10¹¹ Ω·m, 10⁻¹ to 10¹² Ω·m, 10⁻¹ to 10¹³ Ω·m, 10⁻¹ to 10¹⁴ Ω·m, 10⁻¹ to 10¹⁵ Ω·m, 10⁻¹ to 10¹⁶ Ω·m, 1 to 10 Ω·m, 1 to 10² Ω·m, 1 to 10³ Ω·m, 1 to 10⁴ Ω·m, 1 to 10⁵ Ω·m, 1 to 10⁶ Ω·m, 1 to 10⁷ Ω·m, 1 to 10⁸ Ω·m, 1 to 10⁹ Ω·m, 1 to 10¹⁰ Ω·m, 1 to 10¹¹ Ω·m, 1 to 10¹² Ω·m, 1 to 10¹³ Ω·m, 1 to 10¹⁴ Ω·m, 1 to 10¹⁵ Ω·m, 1 to 10¹⁶ Ω·m, 10 to 10² Ω·m, 10 to 10³ Ω·m, 10 to 10⁴ Ω·m, 10 to 10⁵ Ω·m, 10 to 10⁶ Ω·m, 10 to 10⁷ Ω·m, 10 to 10⁸ Ω·m, 10 to 10⁹ Ω·m, 10 to 10¹⁰ Ω·m, 10 to 10¹¹ Ω·m, 10 to 10¹² Ω·m, 10 to 10¹³ Ω·m, 10 to 10¹⁴ Ω·m, 10 to 10¹⁵ Ω·m, 10 to 10¹⁶ Ω·m, 10² to 10³ Ω·m, 10² to 10⁴ Ω·m, 10² to 10⁵ Ω·m, 10² to 10⁶ Ω·m, 10² to 10⁷ Ω·m, 10² to 10⁸ Ω·m, 10² to 10⁹ Ω·m, 10² to 10¹⁰ Ω·m, 10² to 10¹¹ Ω·m, 10² to 10¹² Ω·m, 10² to 10¹³ Ω·m, 10² to 10¹⁴ Ω·m, 10² to 10¹⁵ Ω·m, 10² to 10¹⁶ Ω·m, 10³ to 10⁴ Ω·m, 10³ to 10⁵ Ω·m, 10³ to 10⁶ Ω·m, 10³ to 10⁷ Ω·m, 10³ to 10⁸ Ω·m, 10³ to 10⁹ Ω·m, 10³ to 10¹⁰ Ω·m, 10³ to 10¹¹ Ω·m, 10³ to 10¹² Ω·m, 10³ to 10¹³ Ω·m, 10³ to 10¹⁴ Ω·m, 10³ to 10¹⁵ Ω·m, 10³ to 10¹⁶ Ω·m, 10⁴ to 10⁵ Ω·m, 10⁴ to 10⁶ Ω·m, 10⁴ to 10⁷ Ω·m, 10⁴ to 10⁸ Ω·m, 10⁴ to 10⁹ Ω·m, 10⁴ to 10¹⁰ Ω·m, 10⁴ to 10¹¹ Ω·m, 10⁴ to 10¹² Ω·m, 10⁴ to 10¹³ Ω·m, 10⁴ to 10¹⁴ Ω·m, 10⁴ to 10¹⁵ Ω·m, 10⁴ to 10¹⁶ Ω·m, 10⁵ to 10⁶ Ω·m, 10⁵ to 10⁷ Ω·m, 10⁵ to 10⁸ Ω·m, 10⁵ to 10⁹ Ω·m, 10⁵ to 10¹⁰ Ω·m, 10⁵ to 10¹¹ Ω·m, 10⁵ to 10¹² Ω·m, 10⁵ to 10¹³ Ω·m, 10⁵ to 10¹⁴ Ω·m, 10⁵ to 10¹⁵ Ω·m, or 10⁵ to 10¹⁶ Ω·m).

In some embodiments, the matrix can be removed from the biological sample on a substrate, wherein the biological sample is then further analyzed. For example, a method for analyzing a biological sample can comprises (a) contacting the biological sample with a substrate comprising a plurality of capture probes, wherein at least one capture probe of the plurality of capture probes comprises a capture domain and a spatial barcode; (b) contacting a matrix to a surface of the biological sample; (c) removing the matrix from the surface of the biological sample, thereby providing a further surface of the biological sample; and (d) analyzing an analyte on the further surface of the biological sample to determine presence of the analyte in the biological sample.

The matrix can be removed in any useful manner. In one instance, removal can include the use of an ionization technique that employs an ionization source that is directed to the substrate having the matrix. Non-limiting ionization techniques include matrix-assisted laser desorption ionization (MALDI), desorption electrospray ionization (DESI), secondary ion mass spectrometry (SIMS), liquid extraction surface analysis (LESA), liquid ablation electrospray ionization (LAESI), and the like. For example, such a technique may be useful if the ionization technique is also employed during analysis of one or more analytes. Non-limiting ionization sources include a laser, a plasma, a photon, an arc discharge, an electron ionization source, a chemical ionization source, an electron cyclotron resonance ion source, a particle bombardment source, a field desorption source, a spray ionization source, and the like. After employing an ionization technique, the substrate and the sample may be optionally rinsed (e.g., with a solvent or a solvent system, such as any described herein) and stored (e.g., under cold storage, such as at −80° C.). After storage and prior to analysis, the substrate and the sample may be optionally heated (e.g., to room temperature or physiological temperature, such as about 37° C.) and optionally rinsed (e.g., with a solvent or a solvent system, such as any described herein).

In another instance, removal can include the use of a solvent or a solvent system that is used to rinse the substrate having the matrix. The solvent or solvent system can include any solvent described herein, including combinations or mixtures thereof. In one non-limiting instance, the solvent includes methanol (e.g., cold methanol), which can be used to rinse the surface of the substrate (or the surface of the matrix disposed on the substrate) before mass spectrometry or analysis of the analyte.

Optionally, the sample having the matrix can be stored or maintained for any useful time period and at any useful temperature. Non-limiting time periods can include about 30 minutes, about 1 hour, about 2 hours, about 3 hours, about 4 hours, about 5 hours, about 6 hours, about 8 hours, about 10 hours, about 15 hours, or about 20 hours. Non-limiting temperatures include from about 18° C. to about 25° C., about 20° C. to about 37° C., about 0° C. to about 10° C., about −2° C. to about −8° C., about −28° C. to about −18° C., or about −40° C. to about −80° C.

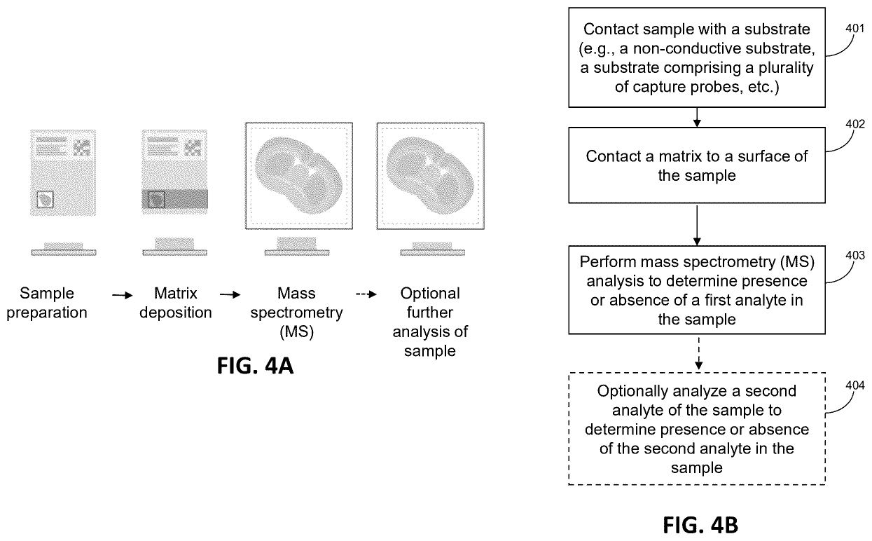

FIG. 4A is a schematic diagram of a non-limiting method that employs a matrix. As can be seen, the method can include sample preparation, matrix deposition, mass spectrometry, and optional further analysis of the sample. Sample preparation can include any useful processes to treat the sample, such as by sectioning, mounting, rinsing, thawing, and/or otherwise processing the sample to provide it on a surface of a substrate. The substrate can be any described herein (e.g., a non-conductive substrate, a conductive substrate, a substrate including a plurality of capture probes, and the like). Matrix deposition can include any process described herein, in which a matrix (optionally in the presence of a solvent or a solvent system) is applied to a surface of the sample disposed on the substrate. MS, which can optionally include MSI, can be performed on the matrix-coated sample. In particular embodiments, MS or MSI can result in removal of the matrix, in which the ionization source is directed to the surface of the matrix-coated sample to ionize the surface, the matrix, and analytes in proximity to the matrix. Further optional processes can be conducted. In one instance, after MS, the sample can be rinsed with a solvent or a solvent system (e.g., any described herein) and then stored (e.g., under any storage conditions described herein). Prior to use, the stored sample can be heated and/or rinsed with a solvent or a solvent system (e.g., any describe herein). In another instance, after MS, the sample can be further analyzed (e.g., by way of spatial transcriptomics).

FIG. 4B shows an exemplary flowchart of the method described in FIG. 4A. As can be seen, the method can include: contacting a sample (e.g., a tissue section) with a substrate (401), in which the substrate can include any described herein, such as a non-conductive substrate, a substrate comprising a plurality of capture probes, etc.; contacting a matrix to a surface of the sample (402), which can generate a mass spectrometry sample surface; and performing MS analysis of the mass spectrometry sample surface to determine presence of an analyte (e.g., a first analyte) in the sample (403). Optionally, the method can further include: analyzing a further analyte (e.g., a second analyte) of the sample to determine presence of the further analyte in the sample (404). Such further analysis can include any described herein (e.g., staining, imaging, spatial transcriptomics, and the like).

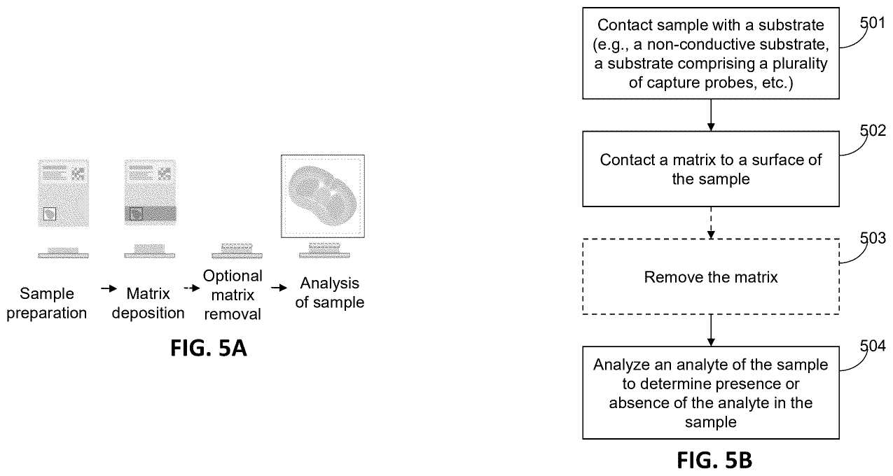

FIG. 5A is a schematic diagram of another non-limiting method that employs a matrix. As can be seen, the method can include sample preparation, matrix deposition, optional matrix removal, and analysis of the sample. Sample preparation can include any useful processes to treat the sample, such as by sectioning, mounting, rinsing, thawing, and/or otherwise processing the sample to provide it on a surface of a substrate. The substrate can be any described herein (e.g., a non-conductive substrate, a conductive substrate, a substrate including a plurality of capture probes, and the like). Matrix deposition can include any process described herein, in which a matrix (optionally in the presence of a solvent or a solvent system) is applied to a surface of the sample disposed on the substrate. Optionally, matrix removal is conducted. Non-limiting processes for matrix removal are described herein.

In one instance, an ionization source, a solvent, or a solvent system is employed to remove the matrix. Other optional processes can be conducted. In one instance, the sample can be rinsed with a solvent or a solvent system (e.g., any described herein) and then stored (e.g., under any storage conditions described herein). Prior to use, the stored sample can be heated and/or rinsed with a solvent or a solvent system (e.g., any describe herein). Analysis of the sample can include any techniques described herein (e.g., staining, imaging, spatial transcriptomics, MS, MSI).

FIG. 5B shows an exemplary flowchart of the method described in FIG. 5A. As can be seen, the method can include: contacting a sample (e.g., a biological sample) with a substrate (501), in which the substrate can include any described herein, such as a non-conductive substrate, a conductive substrate, a substrate comprising a plurality of capture probes, etc.; contacting a matrix to a surface of the sample (502); and analyzing the sample surface to determine presence of an analyte (e.g., a first analyte) in the sample (504). Such analysis can include any methods described herein (e.g., staining, imaging, spatial transcriptomics, MS, MSI, and the like).

Optionally, the method can further include: removing the matrix (503). Other optional operations can include: analyzing a further analyte (e.g., a second analyte) of the sample to determine presence of the further analyte in the sample. Such further analysis can include any method described herein (e.g., staining, imaging, spatial transcriptomics, MS, MSI, and the like).

### (D) Mass Spectrometry Imaging (MSI)

As used herein, “mass spectrometry (MS)” refers to an analytical technique that can be used to measure the mass-to-charge ratio of ions. The results are often presented as a mass spectrum, which is a plot of intensity as a function of the mass-to-change ratio. A mass spectrum is a type of plot of the ion signal as a function of the mass-to-charge ratio, wherein these spectra can be used to determine the elemental or isotopic signature of a sample, the masses of particles and of molecules, and to elucidate the chemical identity or structure of molecules and other chemical compounds. In some embodiments, mass spectrometry analysis comprises laser desorption and ionization and/or electrospray ionization. In some embodiments, mass spectrometry analysis comprises matrix-assisted laser desorption/ionization-mass spectrometry imaging (MALDI-MSI) or matrix-assisted laser desorption electrospray ionization (MALDESI). In some embodiments, mass spectrometry analysis further comprises mass spectrometry imaging.

As used herein, “mass spectrometry imaging (MSI)” is a technique used in mass spectrometry to visualize the spatial distribution of molecules, as biomarkers, metabolites, peptides, or proteins by their molecular masses. For example, after collecting a mass spectrum of a sample (e.g., a biological sample) at one spot, the sample is moved to reach another region, and so on, until the entire sample is scanned. By choosing a peak in the resulting spectra that corresponds to an analyte of interest, the MS data is used to map its distribution across the sample. This results in pictures of the spatially resolved distribution of the analyte where each data set contains a gallery of pictures because any peak in each spectrum can be spatially mapped. In some embodiments, the signal generated by this technique is proportional to the relative abundance of the analyte. In some embodiments, mass spectrometry imaging can include ionization technologies that include, but are not limited to, DESI imaging, MALDI imaging and secondary ion mass spectrometry imaging (SIMS imaging).

As used herein, “MALDI mass spectrometry imaging (MALDI-MSI)” refers to the use of matrix-assisted laser desorption ionization as a mass spectrometry imaging technique in which the sample (e.g., a tissue section) is moved in two dimensions while the mass spectrum is recorded. MALDI-MSI has advantages, such as measuring the distribution of a large amount of analytes at one time without destroying the sample, which make it a useful method in tissue-based studies. In mass spectrometry, matrix-assisted laser desorption/ionization (MALDI) is an ionization technique that uses a laser energy absorbing matrix to create ions from large molecules with minimal fragmentation. In some embodiments, MALDI mass spectrometry can be applied to the analysis of biomolecules (e.g., biopolymers such as DNA, RNA, proteins, peptides, and carbohydrates) and various organic molecules (e.g., polymers, dendrimers, and other macromolecules), which tend to be fragile and fragment when ionized by more conventional ionization methods.

In some embodiments, MALDI mass spectrometry can be a three-step process. First, the sample is mixed with a suitable matrix material and applied to a substrate (e.g., any substrate described herein, including a metal plate, a non-metal plate, a microscopy slide, and the like). Second, a pulsed laser irradiates the sample, triggering ablation and desorption of the biological sample and matrix material. Finally, the analyte molecules are ionized by being protonated or deprotonated in the plume of ablated gases, and then they can be accelerated into a mass spectrometer used to analyze them.

### Sample Preparation

In some embodiments, mass spectrometry imaging is performed with a biological sample (e.g., a tissue section) contacted on a substrate (e.g., microscope slide) and applying a matrix to the biological sample. In some embodiments, the matrix can be applied manually or automatically. In some embodiments, the substrate is then inserted into a mass spectrometer, wherein the mass spectrometer records the spatial distribution of an analyte (e.g., a peptide, protein, or small molecule). In some embodiments, an image processing software can be used to import data from the mass spectrometer to allow visualization and comparison with the optical image of the biological sample.

In some embodiments, the substrate is a conductive substrate. In some embodiments, the conductive substrate is a conductive microscope slide. In some embodiments, the conductive substrate is a metal plate. In some embodiments, the substrate is a non-conductive substrate. In some embodiments, the non-conductive substrate comprises or consists essentially of glass. In some embodiments, the non-conductive substrate is a glass slide. In some embodiments, the non-conductive substrate is a gene expression array. In some embodiments, the non-conductive substrate comprises a plurality of capture probes disposed on a surface of the non-conductive substrate, and wherein at least one capture probe of the plurality of capture probes comprises a capture domain and a spatial barcode. In some embodiments, the capture domain can include a poly(T) sequence. In other embodiments, the substrate is any described herein.

### Image Production and Applications

In some embodiments, mass spectrometry images are constructed by plotting ion intensity versus relative position of the data from the sample. In some embodiments, spatial resolution can highly impact the molecular information gained from the MSI analysis.

In some embodiments, MALDI (matrix-assisted laser desorption/ionization) expands the application of mass spectrometry into the analysis of high molecular weight, non-volatile and thermally labile compounds, such as intact proteins and oligonucleotides.

In some embodiments of the methods described herein, a method for analyzing a biological sample can include (a) contacting the biological sample with a non-conductive substrate; (b) contacting a matrix to a surface of the biological sample, thereby generating a mass spectrometry sample surface; and (c) performing mass spectrometry analysis of the mass spectrometry sample surface to determine presence of an analyte in the biological sample. In some embodiments, the mass spectrometry analysis can be conducted for at least one hour, two hours, three hours, or longer. In some embodiments, the mass spectrometry analysis can be conducted for about 30 minutes, about 1 hour, about 2 hours, about 3 hours, about 4 hours, about 5 hours, about 6 hours, about 8 hours, about 10 hours, about 15 hours, or about 20 hours. In some embodiments, the mass spectrometry analysis can be performed at room temperature (e.g., about 18-25° C.). In some embodiments, the mass spectrometry analysis can be performed at about 18° C., about 19° C., about 20° C., about 21° C., about 22° C., about 23° C., about 24° C., or about 25° C.

In some embodiments of the methods described herein, a method for analyzing a biological sample can include (a) contacting the biological sample with a substrate comprising a plurality of capture probes, wherein at least one capture probe of the plurality of capture probes comprises a capture domain and a spatial barcode; (b) contacting a matrix to a surface of the biological sample; (c) optionally removing the matrix to provide a further surface of the biological sample; and (d) analyzing an analyte on the further surface of the biological sample to determine presence of the analyte in the biological sample.

In some embodiments, mass spectrometry analysis is not performed on the non-conductive substrate after the matrix is contacted to the surface of the biological sample. In some embodiments, the matrix can be removed to provide a further surface of the biological sample. In some embodiments, the further surface can be analyzed to determine a presence of an analyte in the biological sample. In some embodiments, the analyte comprises a RNA, a DNA, or a protein. In some embodiments, the analyte comprises mRNA. In some embodiments, the analyzing comprises spatial transcriptomics. In some embodiments, the spatial transcriptomics comprises hybridizing the analyte to the capture domain, thereby generating a captured analyte.

### (E) Spatial Multimodal Analysis (SMA)

As used herein, “Spatial Multimodal Analysis (SMA)” refers to a multimodal spatial methodology that facilitates ST and MALDI-MSI in a single tissue section with retained specificity and sensitivity. In some embodiments, spatial multimodal analysis includes a method for analyzing a biological sample that comprises (a) contacting the biological sample with a substrate comprising a plurality of capture probes, wherein at least one capture probe of the plurality of capture probes comprises a capture domain and a spatial barcode; (b) contacting a matrix to a surface of the biological sample, thereby generating a mass spectrometry sample surface; (c) performing mass spectrometry analysis on the mass spectrometry sample surface to determine presence of a first analyte in the biological sample, thereby providing a further surface of the biological sample after mass spectrometry analysis; and (d) analyzing a second analyte on the further surface of the biological sample to determine presence of the second analyte in the biological sample.

In some embodiments, the mass spectrometry analysis can include analyzing the first analyte of a plurality of analytes from the mass spectrometry sample surface in a mass spectrometer to determine the presence of the first analyte in the biological sample. In some embodiments, the mass spectrometry analysis further comprises laser desorption and ionization and/or electrospray ionization. In some embodiments, the mass spectrometry analysis comprises matrix-assisted laser desorption/ionization-mass spectrometry imaging (MALDI-MSI) or matrix-assisted laser desorption electrospray ionization (MALDESI). In some embodiments, the mass spectrometry analysis further comprises mass spectrometry imaging.

As used herein, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids. Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, the analyte or the first analyte can include a polymer, a lipid, or a peptide. In some embodiments, the analyte or the second analyte can include a DNA molecule, a RNA molecule, a protein, a small molecule, or a metabolite. In some embodiments, the analyte or the second analyte comprises a RNA, a DNA, or a protein. In some embodiments, the second analyte comprises RNA. In some embodiments, the second analyte is mRNA.

In some embodiments, the analyzing comprises spatial transcriptomics. In some embodiments, the spatial transcriptomics comprises: hybridizing the first analyte or the second analyte (e.g., of a plurality of analytes) to the capture domain, thereby generating a captured analyte; determining (i) all or a part of the sequence of the first analyte or the second analyte, or a complement thereof, (ii) the spatial barcode, or a complement thereof; and using the determined sequence of (i) and (ii) to analyze the first analyte or the second analyte in the biological sample. In some embodiments, the determining step comprises sequencing. In some embodiments, the analyzing step comprises sequencing the spatial barcode.

In some embodiments, the methods can further include, prior to performing the analyzing step, fixing and staining the biological sample. In some embodiments, the fixing comprises methanol fixation. In some embodiments, the staining comprises hematoxylin and/or eosin staining.

In some embodiments, the analysis of an analyte described herein can include, for example, identifying a change or deregulation of the abundance of the analyte, thereby identifying biomarkers for the detection of a disease (e.g., Parkinson's disease). Such changes can be determined or detected in any useful manner, such as by mass spectrometry analysis, spatial transcriptomics, sequencing, or other methodologies described herein. In some embodiments, the methods described herein include identifying the analyte as having increased abundance, over expression, or up regulation, in the location in the biological sample as compared to abundance of the analyte in a corresponding location in a reference, or normal, sample. In some embodiments, the method comprises identifying the analyte as having decreased abundance, under expression or down regulation, in the location in the biological sample as compared to abundance of the analyte in a corresponding location in a reference, or normal, sample.

As used herein, a biomarker can be any appropriate biomarker. In some embodiments, a biomarker can be a nucleic acid (e.g., genomic DNA (gDNA), mRNA, or rRNA (e.g., bacterial 16S rRNA)), a protein, a peptide, or a fragment thereof, (e.g., an enzyme, a cell surface marker, a structural protein, a tumor suppressor, an antibody, a cytokine, a peptide hormone, or an identifiable fragment, precursor, or degradation product of any thereof), a lipoprotein, a cell (e.g., a cell type, for example, in a location indicative of disease), or a small molecule (e.g., an enzymatic cofactor), a hormone (e.g., a steroid hormone or a eicosanoid hormone), or a metabolite. In some embodiments, a biomarker can include an alteration in a nucleic acid (e.g., an insertion, a deletion, a point mutation, a splicing anomaly, and/or methylation), for example, relative to a wildtype or control nucleic acid. In some embodiments, a biomarker can include an alteration in a protein (e.g., an inserted amino acid, a deletion of an amino acid, an amino acid substitution, and/or a post-translational modification (e.g., presence, absence, or a change in, for example, acylation, isoprenylation, phosphorylation, glycosylation, methylation, hydroxylation, amidation, and/or ubiquitinylation)), for example, relative to a control or wild type protein.

### EXAMPLES

### Example 1A—Methods

### Animal Experiment

Four adult male C57Bl/6J mice, 8 weeks old (Charles River, Sulzfeld, Germany) were housed under controlled temperature and humidity (20° C., 53% humidity) with 12 h light/12 h dark cycles. The mice had access to standard food pellets and water ad libitum. Animal work was performed in agreement with the European Council Directive (86/609/EE) and approved by the local Animal Ethics Committee (Stockholms Norra Djurförsöksetiska Nämnd, approval number 3218-2022).

One mouse served as control while three mice were anesthetized with isoflurane (Apoteket, Sweden), pretreated with 25 mg/kg desipramine intraperitoneally (i.p.) (Sigma-Aldrich) and 5 mg/kg pargyline i.p. (Sigma-Aldrich), placed in a stereotaxic frame, and injected over 2 min, with 3 μg of 6-OHDA in 0.01% ascorbate (Sigma-Aldrich) into the median forebrain bundle (MFB) of the right hemisphere. The coordinates for injection were anterior-posterior (AP)—1.1 mm, medial-lateral (ML)—1.1 mm, and dorsal-ventral (DV)—4.8 mm relative to bregma and the dural surface (Paxinos and Franklin, 2001). Post-operative analgesia buprenorphine (Temgesic 0.1 mg/kg) subcutaneously (s.c.) was administered for two days following surgery. Two weeks after unilateral 6-OHDA administration, the lesion was validated by administering the mice with 1 mg/kg apomorphine i.p. (Sigma-Aldrich) and rotational behavior was assessed. Mice were sacrificed, the brains were taken out and stored at −80° C. for further use.

Efforts were taken to minimize the number of animals used and their suffering. Animals were euthanized by decapitation and brains were rapidly dissected out, snap-frozen in dry-ice cooled isopentane for 3 seconds, and stored at −80° C. to minimize postmortem degradation.

### Human Post Mortem Sample

The human postmortem sample was from the caudate putamen level of the brain (coronal sections) of a man who died at 94 years of age. The post-mortem interval until the brain was frozen was 9.25 h. The neuropathological diagnosis was Parkinson's disease in Braak stage 3. The case was obtained from the Harvard Brain Tissue Resource Center at the McLean Hospital (Belmont, MA, USA). Analyses were approved by the local ethical committee (Karolinska Institutet, Stockholm, Sweden, Dnr 2014/1366-31). All experiments were performed in compliance with all relevant ethical regulations.

### Tissue Processing and Sample Preparation

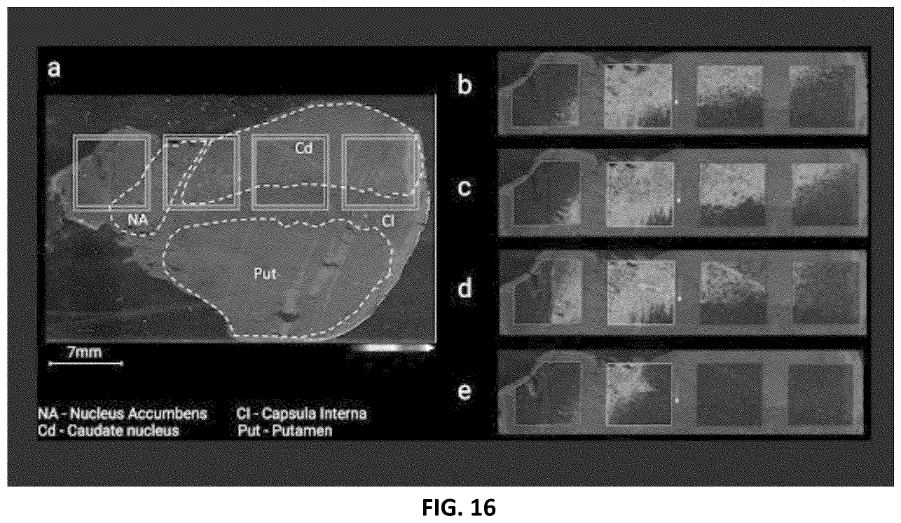

Coronal mouse brain tissue sections, 12 μm thick, were cut at −20° C. using a CM1900 UV cryostat-microtome (Leica Microsystems, Wetzlar, Germany) and subsequently thaw-mounted onto Visium glass slides for SMA and Visium analysis (10× Genomics, Inc.) or conductive indium tin oxide-coated glass slides (Bruker Daltonics, Bremen, Germany) (for MSI analysis). Sections were collected at striatal level (distance from bregma, 0.49 mm, ref) and at the substantia nigra level (distance from bregma, −3.39 mm) for all samples to investigate the substantia nigra in the lesioned mice. The human striatal PD sample was sectioned at 12 μm thickness, and the caudate region was placed over the four printed areas on the Visium slide (FIG. 16A). The prepared slides were stored at −80° C. Sections were desiccated at room temperature for 15 min prior to scanning on a flatbed scanner (Epson Perfection V500, Japan) except for the tissues coated with FMP-10 that were scanned after matrix application. For neurotransmitter analysis, on-tissue chemical derivatization was performed with the FMP-10 reactive matrix. Briefly, a freshly prepared solution of FMP-10 (4.4 mM) in 70% acetonitrile was sprayed onto mouse brain tissue sections and the human tissue sample in 20 passes at 90° C. using a robotic sprayer (TM-Sprayer; HTX Technologies, Chapel Hill, NC) with a flow rate of 80 μL/min, spray head velocity of 1100 mm/min, 2.0 mm track spacing, and 6 psi nitrogen pressure. Tissue sections from the control mouse and from one lesioned mouse were also coated with 9-aminoacridine (9-AA, 5 mg/mL dissolved in 80% methanol) for analysis in negative ionization mode and with 3,5-dihydroxybenzoic acid (DHB, 35 mg/mL dissolved in 50% acetonitrile and 0.2% TFA) for analysis in positive ionization mode. 9-AA was applied using the TM-sprayer (75° C., 6 passes, solvent flow rate of 70 μL/min, spray head velocity of 1100 mm/min, and track spacing of 2.0 mm) and DHB was applied with the same settings except for a nozzle temperature of 95° C. Tissue sections placed on the same glass slide but coated with different matrices were masked using a glass cover slip.

### MALDI-MSI

Tissue sections were imaged at 100 μm lateral resolution using a MALDI-FTICR (Solarix XR 7T-2Ω, Bruker Daltonics, Germany) instrument equipped with a Smartbeam II 2 KHz Nd:YAG laser. The laser power was optimized at the start of each analysis. Spotted red phosphorus was used for external calibration of the methods. Spectra were collected by summing signals from 100 laser shots per pixel. Samples coated with FMP-10 and DHB were analyzed in positive ionization mode. The quadrupole isolation mass-to-charge (m/z) ratio (Q1) was set at m/z 379 (FMP-10) or m/z 150 (DHB), and data were collected over the m/z 150-1050 range and m/z 129-1000, respectively. For the FMP-10 analysis m/z 555.2231 was used as the lock mass and the matrix peak at m/z 273.0394 used as lock mass for internal m/z calibration of the data acquired from the DHB coated sample. Samples coated with 9-AA were analyzed in negative ionization mode over the m/z 107.5-1000 range with a Q1 mass of m/z 120 and m/z 193.0771 was used as lock mass.

### MSI and SMA Metabolomics Data Analysis

The SCiLS Lab API (Bruker Daltonics) was used to create ion images used in downstream analyses. To ensure similar m/z lists among samples with different derivatization matrices, a reference peaklist was chosen to which all other samples of the same derivatization were calibrated, thus having only one list of m/z values per matrix. To compare the performance of Visium and ITO glass, Pearson correlations were computed using the SCilS Lab API (Bruker Daltonics) and the python programming language. UMAPs were performed in the R programming language with a similar script to the spatial transcriptomics UMAP, though changed to accommodate for MSI data.

### Visium

Fresh Frozen samples were cryo-sectioned at 10 μm thickness, placed onto Visium glass slides and stored in −80° C. before processing. Spatial gene expression libraries were generated following 10× Genomic Visium Gene Expression protocol (User Guide, CG000239 Rev F). Libraries were sequenced on NextSeq2000 (Illumina). Length of read 1 was 28 bp and read 2 was 150 bp long.

### Spatial Multimodal Analysis (SMA)

Sections on Visium Gene Expression or Tissue Optimization glass slides (10× Genomics) were desiccated at room temperature for 15 min before application of the reactive matrices. An automated pneumatic sprayer (TM-Sprayer) was used to spray 5 ml of heated reagent over the tissue sections. The nozzle temperature was set at 80° C. for all samples except for those used in the high-resolution imaging experiment (Visium Gene Expression slide), in which the temperature was set at 90° C. The reagents were sprayed over thirty passes using the same parameters as described above and the samples were analyzed without any further incubation. The slides were briefly immersed three times in pre-chilled methanol, followed by storage at −80 until Visium gene expression/tissue optimization processing. Visium Spatial Gene Expression and Tissue Optimization slides, with the exception of the human postmortem sample, were processed according to the corresponding 10× Genomics protocols (User Guide, CG000160 Rev C; User Guide, CG000239 Rev F; and User Guide, CG000238 Rev E). Libraries were sequenced on Nextseq2000 (Illumina). Length of read 1 was 28 bp and read 2 was 150 bp long.

The human postmortem sample was processed according to the RNA-Rescue Spatial Transcriptomics (RRST) protocol (doi: doi.org/10.1101/2022.09.13.507728). The slide was taken out of the −80° C. freezer and placed on a thermocycler pre-heated at 37° C. for 1 minute, followed by immediate fixation in 4% methanol-free formaldehyde (Thermofisher, Catalog number: 28906) solution for 10 minutes at room temperature (RT). After fixation, the slide was washed twice in 1×PBS, heated up at 37° C. for 20 minutes on a thermocycler, cooled down to RT, stained with Hematoxylin and alcoholic Eosin and imaged with a light microscope. Directly after imaging, slides were washed with MQ water, air-dried and placed inside plastic Visium cassettes. Sections were treated with 0.1N HCl for 1 minute at room temperature, and washed in 1×PBS. The Visium Spatial Gene Expression for FFPE reagent kit (10× Genomics, Pleasanton, CA, USA) was used for the downstream steps. Decrosslinking step was skipped, immediately proceeding with probe Pre-hybridization step for 15 minutes at room temperature, followed by Probe Hybridization overnight according to 10× Visium Spatial Gene Expression Reagent Kits for FFPE protocol and the rest of the library preparation (User Guide, CG000407 Rev C). Final libraries were sequenced on Nextseq2000 (Illumina). Length of read 1 and read 2 were 28 base pairs and 50 base pairs, respectively.

### Visium Data Processing

Sequenced libraries were processed using Space Ranger software (version 1.2.1 for standard Visium data and version 1.3.1 for RRST data, 10× Genomics). Reads were aligned to the pre-built human or mouse reference genome provided by 10× Genomics (GRCh38 for human data or mm 10 for mouse data, version 32, Ensembl 98), which includes a GTF file, a FASTA file and a STAR index.

### Visium, RRST and SMA Transcriptomics Data Analysis

Processing and analysis of spatial transcriptomics data obtained with either standard Visium, RRST or SMA was performed using R (v4.1.3), the single-cell genomics toolkit Seurat and the spatial transcriptomics toolkit STUtility. The Hematoxylin and Eosin images were manually annotated based on tissue morphology and dopamine expression using the interactive application Loupe Browser provided by 10× Genomics. Mouse striatum and substantia nigra hemispheres were categorized into two groups: “intact” and “lesioned.” The human tissue section was categorized into two groups (“Dop+” and “Dop−”) based on the dopamine expression pattern detected by the MSI step of the SMA protocol. Filtered count matrix from Space Ranger output were used for downstream analysis with additional filters. In particular, spatially barcoded features below sectioning or mounting artifacts were annotated using Loupe Browser and removed using “SubsetSTData” function in STUtility; spatially barcoded features with more than 38% mitochondrial genes or less than 50 unique genes were removed using the same STUtility function; hemoglobin-coding, riboprotein-coding and Malat1 genes were removed from the dataset as well. Gene-gene scatter plots comparing detection rates were created as follows: raw expression matrices were extracted for each data type (RRST or Visium Gene Expression for FFPE) and the detection rates were estimated for each gene as the proportion of spatially barcoded features with detected UMI counts. Pearson R scores and p-values were calculated using the continuous=“cor” argument in ggpairs function or the corrplot function of the GGally or corrplot R packages, respectively. The percentage of genes across technical and biological conditions was calculated supplying a list of all the genes with count higher than 1 for each condition to the ggVennDiagram function of the ggVennDiagram R package. After filtering out spatially barcoded features and genes as described above, the data were normalized and subjected to a basic analysis workflow using functions from the Seurat R package. Normalization and variance stabilization of the data was done using the SCTransform function, followed by dimensionality reduction by PCA (RunPCA). Data were integrated with RunHarmony function from the harmony R package using group.by.vars=“Sample.ID”, assay.use=“SCT” and reduction=“pca” as parameters. A shared nearest neighbor (SNN) graph was constructed based on the first 30 principal components (FindNeighbors). Finally, a Uniform Manifold Approximation and Projection (UMAP) embedding was computed based on the first 30 principal components (RunUMAP) followed by graph-based clustering (FindClusters). Genes up- or down-regulated in the intact hemisphere of the mouse samples or the intact area of the human brain were detected by calculating differential expression between the annotated region and the background (remaining spatially barcoded features) with an adjusted p-value threshold of 0.01 using the FindAllMarkers function. The module scores for the intact mouse hemispheres or human brain area were calculated supplying the list of all the up-regulated genes in the respective regions to the AddModuleScore function.

### Example 1B—Spatial Multimodal Analysis (SMA) Workflow

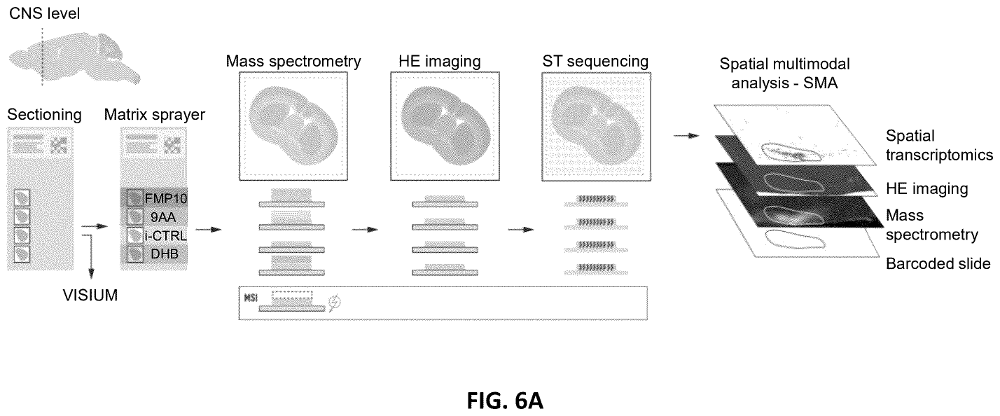

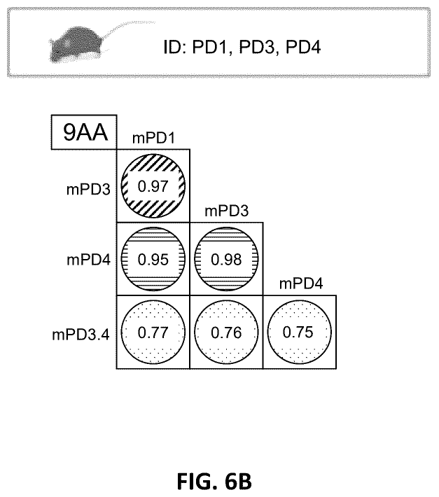

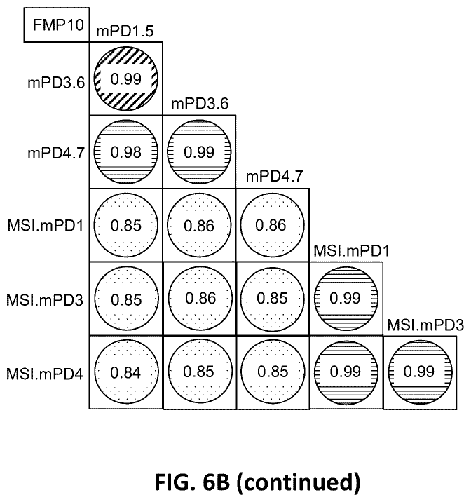

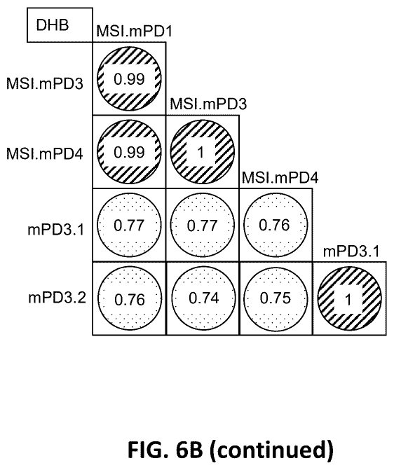

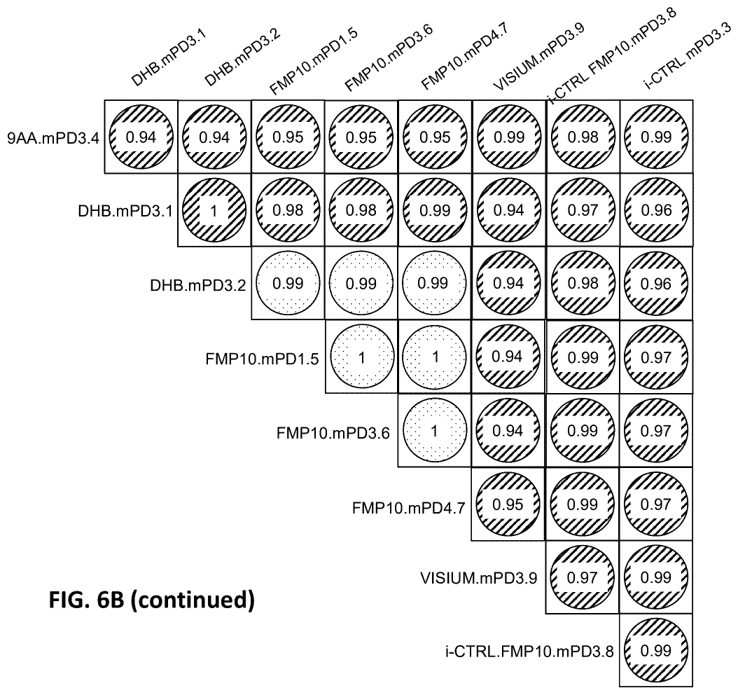

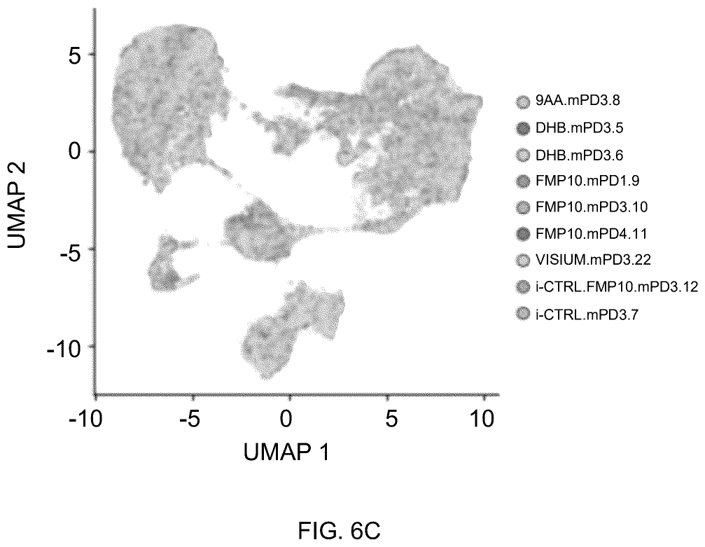

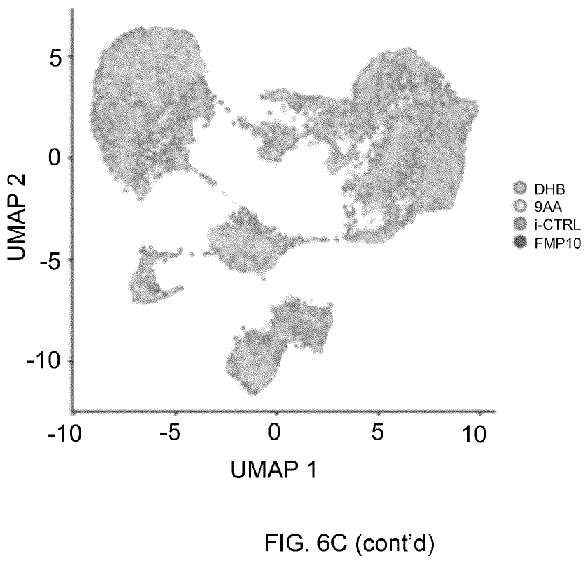

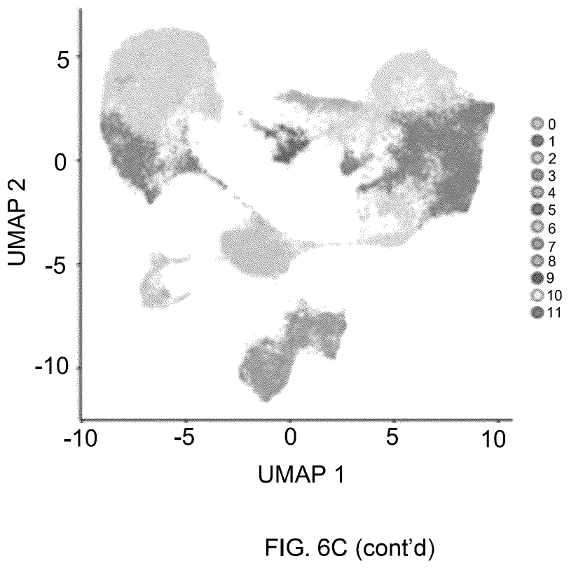

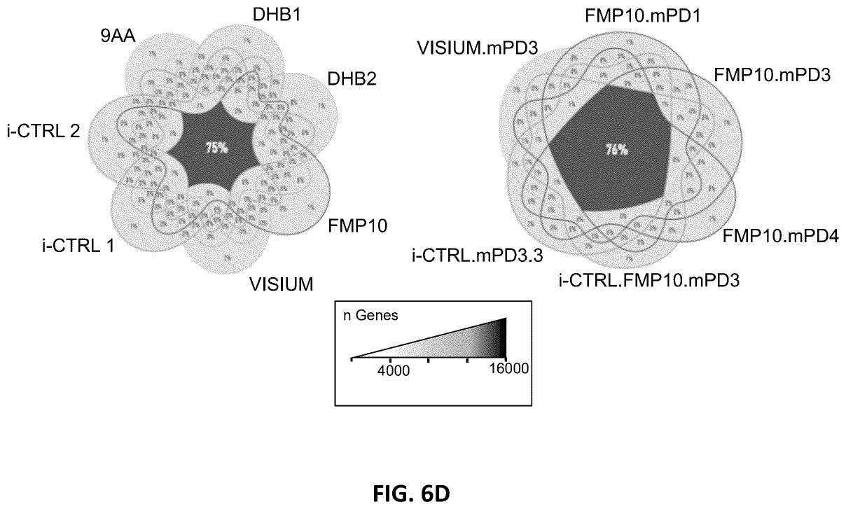

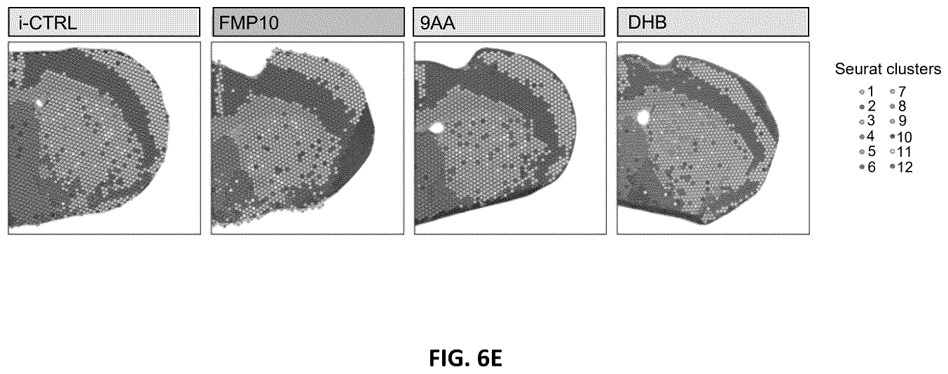

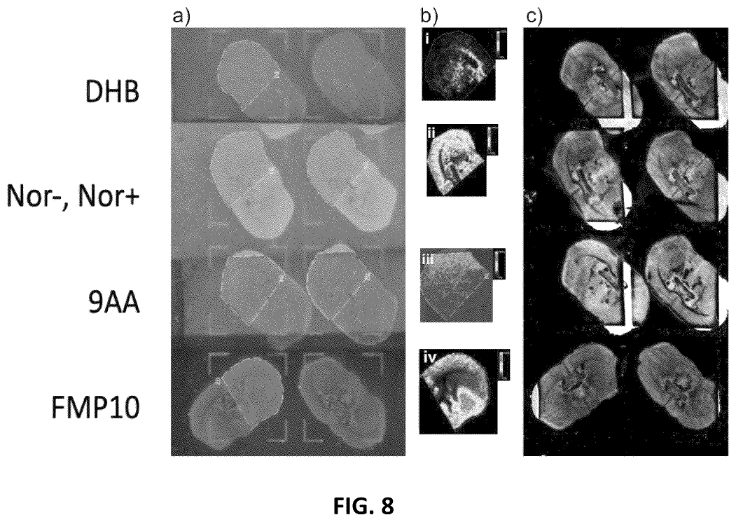

In a non-limiting example, SMA workflow can be composed of four steps: i) sectioning non-embedded snap-frozen samples onto non-conductive barcoded gene expression arrays; ii) mass spectrometry imaging; iii) hematoxylin and eosin (HE) staining and light microscopy imaging; and finally, iv) spatial transcriptomics (FIG. 6A). In order to test the feasibility of the SMA method, an RNA quality check assay was performed using mouse brain tissue sections. Sections were mounted on slides coated with polydT probes and sprayed (nozzle temperature >80° C.) with four different MALDI matrices: 9-Aminoacridine (9-AA), 2,5-dihydroxybenzoic acid (DHB), Norharmane, and 2-fluoro-1-methyl pyridinium (FMP-10), the latter being a matrix recently developed to comprehensively map neurotransmitters in the brain (FIG. 8). Tissue sections were then imaged at 100 μm resolution using MALDI-FTICR (Bruker), and spectra were collected during approximately three hours at room temperature. After MSI, H&E staining, and light microscopy imaging, the presence of mRNA transcripts in the mouse brain tissue sections was determined by reverse transcription in the presence of fluorescent nucleotides. The results showed strong MALDI MSI signals for the four investigated matrices in line with the conventional conductive indium tin oxide-coated glass slides on non-matched mouse brains. The subsequent RNA quality analysis demonstrated a distinct spatial pattern, indicating that mRNA is still present after mass spectrometry imaging (FIG. 8).

### Example 2—Quantify and Demonstrate Reproducibility of SMA

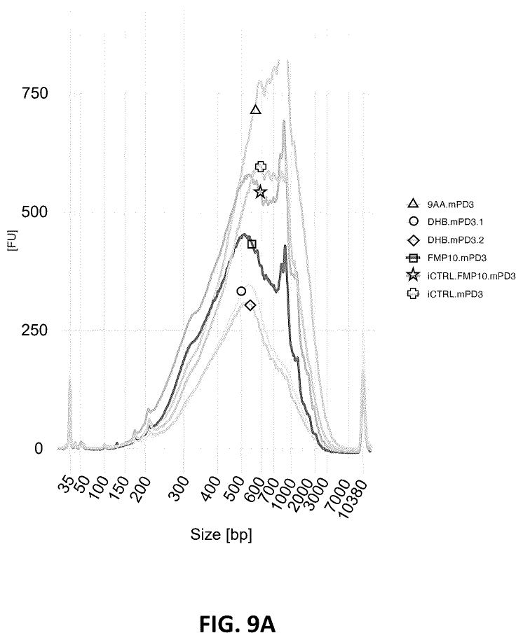

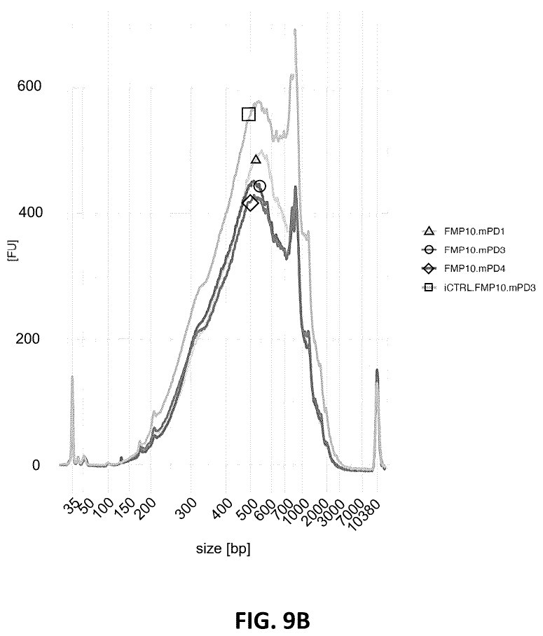

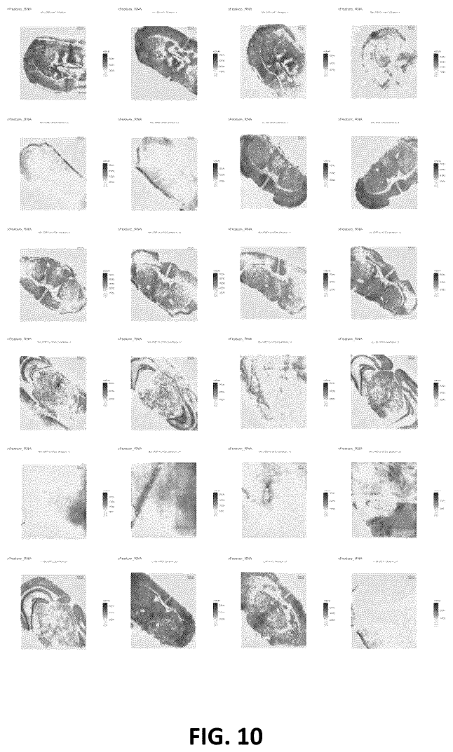

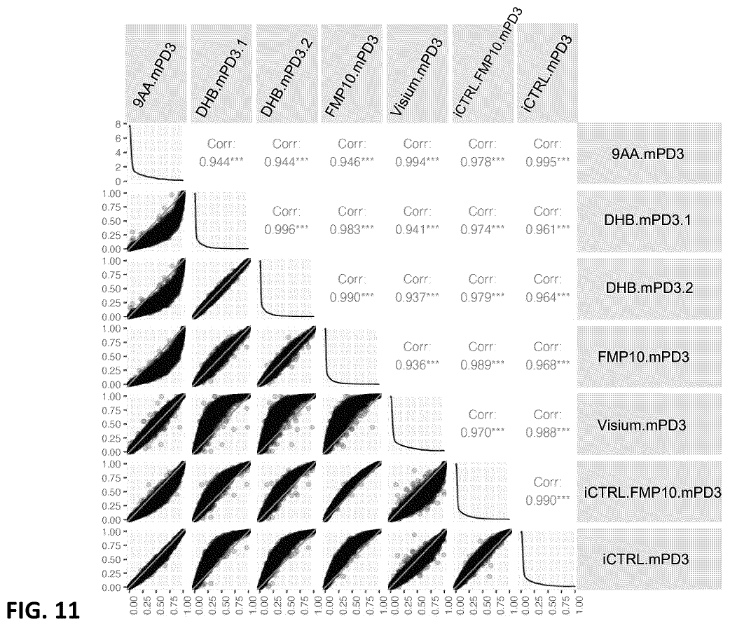

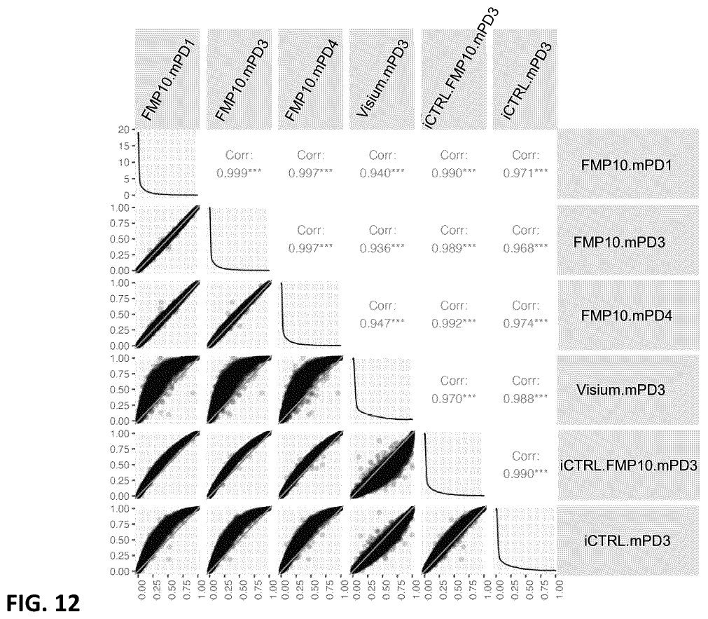

In a non-limiting example, the reproducibility of the SMA method was quantified and demonstrated. For this purpose, the experiments were repeated using barcoded oligonucleotide slides (Visium Gene Expression slides), which enable the detection and quantification of individual captured transcripts by sequencing. Consecutive brain sections were used from three different mice, imaged with three different MALDI matrices (DHB, 9AA and FMP10) and compared the gene expression or MALDI MSI data with matching tissue sections analyzed using the Visium Gene Expression or MSI protocols, respectively. The analysis of cDNA electrophoresis curves and non-filtered ST data suggested that performing MSI before ST does not result in increased RNA degradation nor diffusion (FIGS. 9A-9B and 10). It was demonstrated that both gene expression and small molecule profiles highly correlate to the reference data produced using the two corresponding methods individually: all correlations are above 0.74 and p-values lower than 0.05 (FIGS. 6B, 11, and 12).

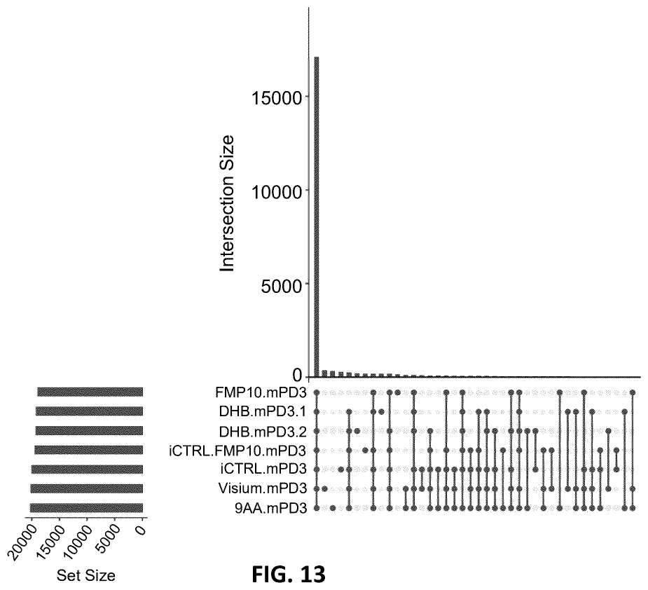

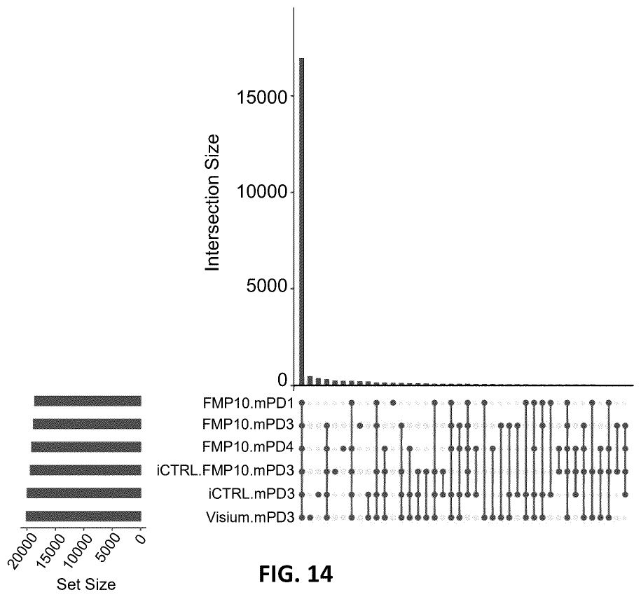

The efficiency of the SMA method was also measured as the percentage of detectable genes compared to the standard Visium Gene Expression protocol. Results showed that at least 75% of the genes were detected across all technical and biological conditions tested (FIGS. 6D, 13, and 14). Finally, the projection of clustered transcriptomics data on Uniform Manifold Approximation and Projection (UMAP) and spatial maps showed that both data modalities integrate well across different experimental conditions, and that the spatial patterns of the detected genes are highly conserved across experimental conditions (FIGS. 6C and 6E).

### Example 3—Demonstrate SMA Applicability Using Clinically Relevant Model Specimens

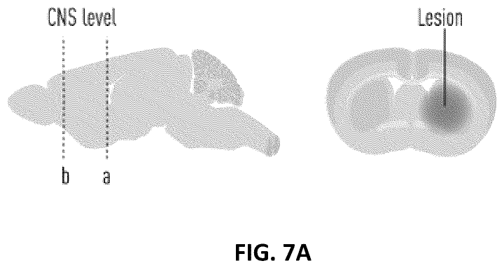

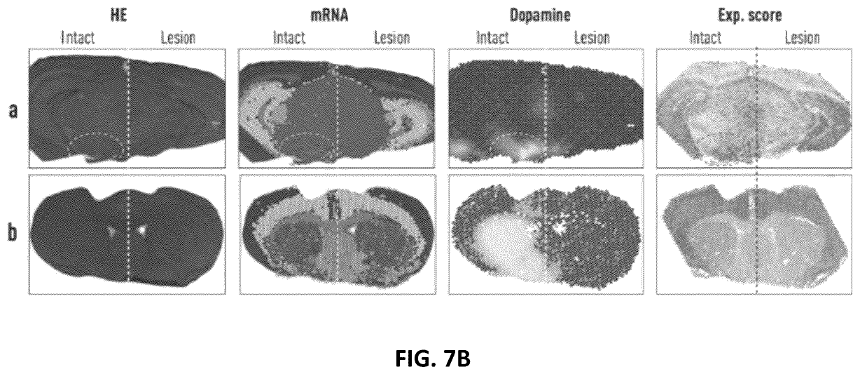

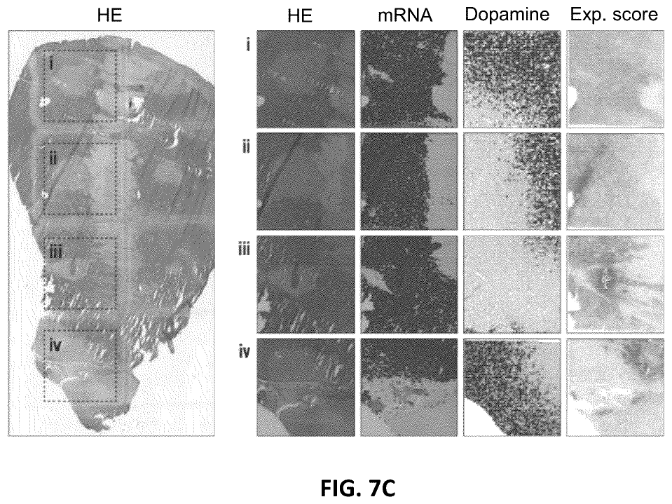

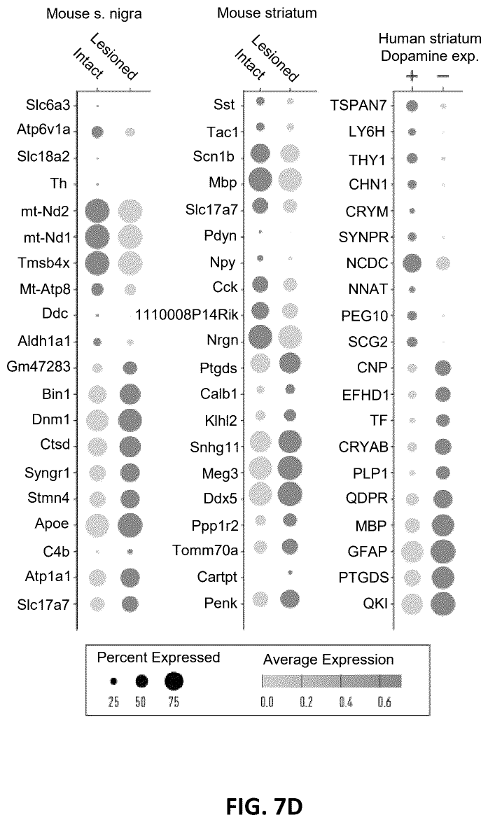

In a non-limiting example, SMA's applicability was demonstrated using clinically relevant model specimens. Parkinson's disease (PD) is the most common neurodegenerative disorder after Alzheimer's disease, and it is characterized by the loss of dopaminergic neurons within the substantia nigra pars compacta (SNpc), which contains neurons that project to the dorsal putamen of the striatum. Here, it was aimed to capture gene expression and corresponding neurotransmitters at two brain regions (SNpc and striatum) of three different unilateral 6-hydroxydopamine-lesioned mice. 6-hydroxydopamine is a neurotoxin that if injected into the medial forebrain bundle causes near-complete loss of nigral dopaminergic neurons and dopamine (DA) levels within the nigrostriatal pathway (FIG. 7A). As expected, SMA predominantly detected dopamine in the intact striatum and SNpcin, as contrasted to the lesioned contralateral areas. Using the spatial transcriptomics data produced with SMA, the gene expression modules expressed by the intact hemisphere both in the SNpc and striatum were characterized (FIGS. 7B and 7D). As expected, among the genes which were higher in the intact SNpc, TH, which encodes the rate-limiting enzyme in dopamine synthesis, and slc6a3, which encodes the dopamine transporter, were found, which demonstrates that the present results can be attributed to the 6-OHDA-induced destruction of dopaminergic neurons (FIGS. 6A and 7D).

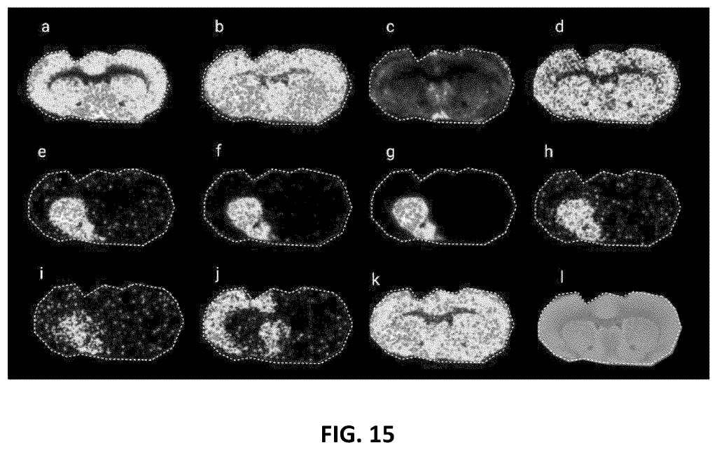

Furthermore, the localization of multiple neurotransmitters and metabolites were detected with high accuracy, namely: dopamine, taurine, 3-methoxytyramine (3-MT), 3,4-dihydroxy-phenylacetaldehyde (DOPAL), 3,4-dihydroxyphenylacetic acid (DOPAC), norepinephrine, serotonin, histidine, tocopherol and GABA together with an image of the scanned tissue (FIG. 15).

### Example 4—SMA Methods Against Human PD Postmortem Material

In a non-limiting example, the SMA methodology was tested against a human PD postmortem material by analyzing striatal brain tissue sections (FIG. 16A). Since MSI is less compatible with the analysis of formalin-fixed material, fresh frozen material was used. Neurotransmitters and gene expression were comprehensively measured over a 2.4×0.5 cm tissue section. To address expected RNA degradation in the postmortem tissue, a protocol that enables gene expression measurements even on fresh frozen tissues with low quality RNA was used. In short, after mass spectrometry imaging, a transcriptome-wide probe panel was used, wherein rather than depending on the poly-A tail, it hybridizes to the mRNA. The spatial distribution of DA, 3-MT, serotonin, and norepinephrine (FIG. 16B-16E) showed results, where higher levels were observed in the medial division of the ventral caudate nucleus. At the same time, the capsula interna displayed lower concentrations of the screened molecules. Using the MALDI-MS images of DA distribution, the gene expression modules associated with the intact (Dop+) and denervated (Dop−) areas were defined. 1612 significantly dysregulated genes were found, of which 1243 were upregulated and 369 downregulated in the Dop+ area, respectively (p-value adjusted <0.01) (FIG. 7C). Interestingly, 31 of the upregulated and four of the downregulated genes were detected both in the human and the mouse datasets. As for the mouse striatum, the spatial projection of the module score for the genes upregulated in the intact area showed a pattern in line with the localization of the neurotransmitters in the human striatum.

Overall, described herein is a general approach to spatially match analytes with gene expression within a tissue section, thereby facilitating the precise mapping of analytes (e.g., neurotransmitters or others) in a cellular neighborhood. This approach has relevance for the investigation of neurological diseases (e.g., PD), but the impact could be broader in oncology or other diseases. Indeed, the spatial multimodal characterization of small molecule drugs with overlapping histology and spatial gene expression information can provide new mechanistic insights into the dynamic crosstalk that regulates the tumor microenvironment and drives the response to treatment. Gene expression can efficiently inform about cell type composition but also infer genome integrity, which can be of interest to match tumor clones with drug efficacy. This study builds further on the notion of the importance of studying analytes in a tissue context providing a new level of multimodality.

What is claimed is:

1. A method for analyzing a biological sample, the method comprising:
(a) contacting the biological sample with a substrate comprising a plurality of capture probes, wherein a capture probe of the plurality of capture probes comprises: (i) a spatial barcode and (ii) a capture domain;
(b) contacting a matrix to a surface of the biological sample, thereby generating a mass spectrometry sample surface;
(c) performing mass spectrometry analysis on the mass spectrometry sample surface to determine presence of a first analyte in the biological sample, thereby providing a further surface of the biological sample after mass spectrometry analysis; and
(d) analyzing a second analyte on the further surface of the biological sample to determine presence of the second analyte in the biological sample.

2. The method of claim 1, wherein the mass spectrometry analysis comprises analyzing the first analyte of a plurality of analytes from the mass spectrometry sample surface in a mass spectrometer to determine the presence or abundance of the first analyte in the biological sample, optionally wherein the mass spectrometry analysis further comprises:
laser desorption and ionization and/or electrospray ionization, or
the mass spectrometry analysis comprises matrix-assisted laser desorption/ionization-mass spectrometry imaging (MALDI-MSI) or matrix-assisted laser desorption electrospray ionization (MALDESI).

3. The method of claim 1, wherein the mass spectrometry analysis further comprises mass spectrometry imaging, optionally wherein the mass spectrometry analysis is conducted for at least one hour, two hours, three hours, or longer, optionally wherein the mass spectrometry analysis is performed at about 18-25° C.

4. The method of claim 1, wherein the second analyte comprises RNA, optionally mRNA, DNA, or a protein, optionally wherein the analyzing comprises spatial transcriptomics.

5. The method of claim 4, wherein the spatial transcriptomics comprises:
hybridizing the second analyte to the capture domain, thereby generating a captured analyte;
determining (i) the sequence of the second analyte, or a complement thereof, and (ii) the spatial barcode, or a complement thereof; and
using the determined sequence of (i) and (ii) to analyze the second analyte in the biological sample.

6. The method of claim 5, wherein the determining step comprises sequencing.

7. The method of claim 1, further comprising, prior to performing the analyzing step, fixing and/or staining the biological sample, optionally wherein the fixing comprises methanol fixation, optionally wherein the staining comprises hematoxylin and/or eosin staining.

8. The method of claim 1, wherein the substrate is a non-conductive substrate.

9. The method of claim 8, wherein the non-conductive substrate is glass.

10. The method of claim 1, wherein the biological sample is a tissue section.

11. The method of claim 10, wherein the tissue section is a fixed tissue section or a fresh-frozen tissue section.

12. The method of claim 1, wherein the capture probe further comprises one or more functional domains, a cleavage domain, a unique molecular identifier, or a combination thereof.

13. The method of claim 1, wherein the matrix comprises: 9-aminoacridine (9-AA), 2,5-dihydroxybenzoic acid (DHB), norharmane, and 2-fluoro-1-methyl pyridinium (FMP-10), or a combination thereof.

14. The method of claim 1, wherein the matrix comprises: benzoic acid, hydroxybenzoic acid, dihydroxybenzoic acid, terephthalic acid, naphthoic acid, cinnamic acid, hydroxycinnamic acid, picolinic acid, benzamide, aniline, acridine, quinoline, naphthalene, anthracene, acetophenone, pyridine, coumarin, norharmane, or a combination thereof.

15. The method of claim 1, wherein contacting the matrix in (b) comprises providing the matrix with a solvent.

16. The method of claim 15, wherein the solvent comprises acetonitrile, methanol, ethanol, propanol, water, acetone, chloroform, or acetonitrile mixed with (trifluoroacetic acid) TFA.

17. The method of claim 15, further comprising rinsing the mass spectrometry sample surface with a further solvent.

18. The method of claim 1, further comprising removing the matrix from the biological sample.

19. The method of claim 1, further comprising imaging the biological sample.

20. The method of claim 1, wherein the capture domain comprises a poly(T) sequence.
